SEQUENCE LISTING (24 sequences)

SEQ ID NO: 1 - DNA, 1497 bp, Mus musculus
  CDS (1)...(1497): Mouse DGAT1 coding sequence
atgggcgaccgcggaggcgcgggaagctctcggcgtcggaggaccggctcgcgggtttccgtccagggtggtagtgggcccaaggtagaagaggacgaggtgcgagacgcggctgtgagccccgacttgggcgccgggggtgacgcgccggctccggctccggctccagcccatacccgggacaaagacgggcggaccagcgtgggcgacggctactgggatctgaggtgccatcgtctgcaagattctttgttcagctcagacagtggtttcagcaattatcgtggtatcctgaattggtgtgtggtgatgctgatcctgagtaatgcaaggttatttttagagaaccttatcaagtatggcatcctggtggatcctatccaggtggtgtctctgtttttgaaggacccctacagctggcctgccccatgcgtgattattgcatccaatatttttgttgtggctgcatttcagattgagaagcgcctggcagtgggtgccctgacagagcagatggggctgctgctacatgtggttaacctggccacaatcatttgcttcccagcagctgtggccttactggttgagtctatcactccagtgggttccgtgtttgctctggcatcatactccatcatgttcctcaagctttattcctaccgggatgtcaacctgtggtgccgccagcgaagggtcaaggccaaagctgtctctacagggaagaaggtcagtggggctgctgcccagcaagctgtgagctatccagacaacctgacctaccgagatctctattacttcatctttgctcctactttgtgttatgaactcaactttcctcggtccccccgaatacgaaagcgctttctgctacgacgagttcttgagatgctcttttttacccagcttcaagtggggctgatccaacagtggatggtccctactatccagaactccatgaagcccttcaaggatatggactattcacggatcattgagcgtctcttaaagctggcggtccccaaccatctgatctggcttatcttcttctattggtttttccactcctgtctcaatgctgtggcagagcttctgcagtttggagaccgcgagttctacagagattggtggaatgctgagtctgtcacctacttttggcagaactggaatatccccgtgcacaagtggtgcatcagacacttctacaagcctatgctcagacatggcagcagcaaatgggtggccaggacaggagtatttttgacctcagccttcttccatgagtacctagtgagcgttcccctgcggatgttccgcctctgggcattcacagccatgatggctcaggtcccactggcctggattgtgggccgattcttccaagggaactatggcaatgcagctgtgtgggtgacactcatcattgggcaaccggtggctgtgctcatgtatgtccacgactactacgtgctcaactacgatgccccagtgggggtatga

SEQ ID NO: 2 - DNA, 1467 bp, Homo sapiens
  CDS (1)...(1467): Homo sapiens diacylglycerol O-acyltransferase homolog 1
atgggcgaccgcggcagctcccggcgccggaggacagggtcgcggccctcgagccacggcggcggcgggcctgcggcggcggaagaggaggtgcgggacgccgctgcgggccccgacgtgggagccgcgggggacgcgccagccccggcccccaacaaggacggagacgccggcgtgggcagcggccactgggagctgaggtgccatcgcctgcaggattctttattcagctctgacagtggcttcagcaactaccgtggcatcctgaactggtgtgtggtgatgctgatcttgagcaatgcccggttatttctggagaacctcatcaagtatggcatcctggtggaccccatccaggtggtttctctgttcctgaaggatccctatagctggcccgccccatgcctggttattgcggccaatgtctttgctgtggctgcattccaggttgagaagcgcctggcggtgggtgccctgacggagcaggcgggactgctgctgcacgtggccaacctggccaccattctgtgtttcccagcggctgtggtcttactggttgagtctatcactccagtgggctccctgctggcgctgatggcgcacaccatcctcttcctcaagctcttctcctaccgcgacgtcaactcatggtgccgcagggccagggccaaggctgcctctgcagggaagaaggccagcagtgctgctgccccgcacaccgtgagctacccggacaatctgacctaccgcgatctctactacttcctcttcgcccccaccttgtgctacgagctcaactttccccgctctccccgcatccggaagcgctttctgctgcgacggatccttgagatgctgttcttcacccagctccaggtggggctgatccagcagtggatggtccccaccatccagaactccatgaagcccttcaaggacatggactactcacgcatcatcgagcgcctcctgaagctggcggtccccaatcacctcatctggctcatcttcttctactggctcttccactcctgcctgaatgccgtggctgagctcatgcagtttggagaccgggagttctaccgggactggtggaactccgagtctgtcacctacttctggcagaactggaacatccctgtgcacaagtggtgcatcagacacttctacaagcccatgcttcgacggggcagcagcaagtggatggccaggacaggggtgttcctggcctcggccttcttccacgagtacctggtgagcgtccctctgcgaatgttccgcctctgggcgttcacgggcatgatggctcagatcccactggcctggttcgtgggccgctttttccagggcaactatggcaacgcagctgtgtggctgtcgctcatcatcggacagccaatagccgtcctcatgtacgtccacgactactacgtgctcaactatgaggccccagcggcagaggcctga

SEQ ID NO: 3 - DNA, 1497 bp, Rattus norvegicus
  CDS (1)...(1497): s norvegicus diacylglycerol O-acyltransferase 1 coding sequence
atgggcgaccgcggaggcgcgggaagctctcggcgtcgcaggaccggctcgcgggtttccgtccagggaggtagtgggcccaaggtagaagaggacgaggtgcgagaagcggctgtgagccccgacttgggcgccgggggtgacgcgccggctccggctccggctccagcccatacccgggacaaagaccggcagaccagcgtgggcgacggccactgggagctgaggtgccatcgtctgcaagactctttgttcagctcagacagcggtttcagcaattaccgtggtatcctgaattggtgcgtggtgatgctgatcctgagtaatgcaaggttatctttagagaatcttatcaagtatggcatcctggtggatcccatccaggtggtgtctctgtttctgaaggacccctacagctggcctgccccatgcttgatcattgcatccaatatctttattgtggctacatttcagattgagaagcgcctgtcagtgggtgccctgacagagcagatggggctgctgctacatgtggttaacctggccacaattatctgcttcccagcagctgtggccttactggttgagtctatcactccagtgggttccctgtttgctctggcatcatactccatcatcttcctcaagctttcttcctaccgggatgtcaatctgtggtgccgccagcgaagggtcaaggccaaagctgtgtctgcagggaagaaggtcagtggggctgctgcccagaacactgtaagctatccggacaacctgacctaccgagatctctattacttcatctttgctcctactttgtgttatgaactcaactttcctcgatccccccgaatacgaaagcgctttctgctacggcgggttcttgagatgctctttttcacccagcttcaagtggggctgatccagcagtggatggtccctactatccagaactccatgaagcccttcaaggacatggactattcacgaatcattgagcgtctcttaaagctggcggtccccaaccatctgatatggctcatcttcttctattggcttttccactcatgtctcaatgctgtggcagagctcctgcagtttggagaccgcgagttctacagggactggtggaatgctgagtctgtcacctacttttggcagaactggaatatccccgtgcacaagtggtgcatcagacacttttacaagcctatgctcagactgggcagcaacaaatggatggccaggactggggtcttttgggcgtcagctttctttcatgagtacctagtgagcattcccctgaggatgttccgcctttgggcattcacagcaatgatggctcaggtcccactggcctggattgtgaaccgcttcttccaagggaactatggcaatgcagctgtgtgggtgacactcatcattgggcaaccggtggctgtgctcatgtatgtccacgactactacgtgctcaactatgatgccccagtgggggcatga

SEQ ID NO: 4 - DNA, 1470 bp, Bos taurus
  CDS (1)...(1470): Bos taurus dgat coding sequence
atgggcgaccgcggcggcgcgggcggctcccggcgccggaggacggggtcgcggccttcgatccagggcggcagtgggcccgcggcagcggaagaggaggtgcgggatgtgggcgccggaggggacgcgccggtccgggacacagacaaggacggagacgtagacgtgggcagcggccactgggacctgaggtgtcaccgcctgcaggattccctgttcagttctgacagtggcttcagcaactaccgtggcatcctgaattggtgtgtggtgatgctgatcttaagcaacgcacggttatttctagagaacctcatcaagtatggcatcctggtggaccccatccaggtggtgtctctgttcctgaaggacccctacagctggccagctctgtgcctggtcattgtggccaatatctttgccgtggctgcgttccaggtggagaagcgcctggccgtgggagctctgacggagcaggcggggctgctgctgcacggggtcaacctggccaccattctctgcttcccagcggccgtggcctttctcctcgagtctatcactccagtgggctccgtgctggccctgatggtctacaccatcctcttcctcaagctgttctcctaccgggacgtcaacctctggtgccgagagcgcagggctggggccaaggccaaggctgctttggcaggtaaggcggccaacgggggagctgcccagcgcaccgtgagctaccccgacaacctgacctaccgcgatctctactacttcctcttcgcccccaccctgtgctacgagctcaacttcccccgctccccccgcatccgaaagcgcttcctgctgcggcgactcctggagatgctgttcctcacccagctccaggtggggctgatccagcagtggatggtcccggccatccagaactccatgaagcccttcaaggacatggactactcccgcatcgtggagcgcctcctgaagctggcggtccccaaccacctcatctggctcatcttcttctactggctcttccactcctgcctgaacgccgtggctgagctcatgcagtttggagaccgcgagttctaccgggactggtggaactccgagtccatcacctacttctggcagaactggaacatccctgttcacaagtggtgcatcagacacttctacaagcccatgctccggcggggcagcagcaagtgggcagccaggacggcagtgtttctggcctccgccttcttccacgagtacctggtgagcatccccctgggaatgttccgcctctgggccttcaccggcatgatggcgcagatcccgctggcctggatagtgggccgcttcttccgcggcaactacggcaacgcggccgtgtggctgtcactcatcatcgggcagccggtggccgtcctgatgtacgtccacgactactacgtgctcaaccgtgaggcgccggcagccggcacctga

SEQ ID NO: 5 - DNA, 1497 bp, Caenorhabditis elegans
  CDS (1)...(1497): Caenorhabditis elegans diacylglycerol acyltransferase coding sequence
atgcaaatgcgtcaacaaacgggacgacggcggcgtcagccttcggaaacatctaatggttctttggcttccagtagacgctcctcatttgcacaaaatggtaattcgtcaaggaaaagttcagaaatgagagggccttgcgagaaagtggtacatactgctcaagattcattgttttcgacgagttctggatggacaaatttccgtggattcttcaatttgtctattttacttttggtactttcaaatggacgcgtggcacttgaaaatgtgatcaaatatggtattttgataacaccccttcagtggatctcaacgtttgttgagcatcactactcaatttggagctggccaaatcttgctctcatcctatgctcaaatattcagattctctcggtttttggaatggaaaaaattcttgaacgtggatggcttggaaacggattcgctgcagtgttctacacctcgcttgtgattgcacatctgacaattccagttgtggtcactcttacccacaaatggaagaatcctttgtggtcagtcgtaatgatgggtgtttatgttattgaagctctcaaattcatctcatatggccacgtcaactactgggctcgtgatgctcggcgaaaaatcacagagctcaaaacacaagtcaccgatttggcaaagaaaacatgtgatccgaaacaattttgggatttgaaagatgaattatcaatgcatcagatggctgctcaatatcctgccaatttgacactttccaatatctactacttcatggctgcaccaacattgtgctacgaattcaaatttccaagattgttgcgaattcggaagcactttttgattaaaagaaccgtggagcttatctttctatcgtttttgatagctgcacttgttcaacaatgggttgttccgactgtccgaaatagtatgaaacctttaagtgaaatggaatactctagatgtttggaacgactcttgaaacttgcaattccaaatcatctcatctggcttctattcttctacacattcttccattcatttttgaacttgatcgccgagctgcttcgatttgccgatcgtgagttctacagagacttttggaatgcagagacgataggatatttctggaaatcatggaacatcccagttcaccgatttgctgttcgccacatctacagtccaatgatgcgtaacaatttctcaaaaatgagcgcattcttcgttgtgttcttcgtgtcggcattcttccatgaatatctggtttctgtgccattaaagattttccgattgtggtcctactatggaatgatgggacaaattcctctatccattatcactgataaagtggtgagaggtggacgtacaggaaacatcatcgtctggctctcactgattgttggccaacctcttgcaattctcatgtacggacatgattggtacattttgaactttggtgtttcagcagttcaaaaccaaaccgttggtatttga

SEQ ID NO: 6 - DNA, 1698 bp, Drosophila melanogaster
  CDS (1)...(1698): Drosophila melanogaster clone 12 acyl coenzyme Adiacylglycerol acyltransferase coding sequence
atgactaccaataaggatccccaagataaggagcccgggaaagcagaacaaccgaccaagaatagcggatccagcggagtgggtatcatgaagcgcttgagaagatcggcgtccgccacagagcataatcttagcagtctgcgaaaccgcaagtcaacacaaaatctattcgatcagcacgggaatcccatagatctgcgacagtatcgtaaagttttggataaggatgaaaatggtaatggaaccaacggatccgagaagaagcttagatacaggagaacacaaagtgtgactcgtgctgaggagatttccaataaagaggagaagcagagaagagctcagcctggcagaccaatccatcggccaagagattctctgttttcttggagctctggatttaccaatttttctggactggtgaactggggatttctactgctctgcattggaggtctgcgtttgggcttggagaatctcctaaagtatggcattcgcatcaatccactggattggttcttcttcataagcggacacaacgaaggcgaaggacataacgccctaatcctgagcatttactctttagtgcatatctcgctctgtttggctgtggagaagggtctagccatggaaataattgcagagggcttgggcttgttcatccagatagtgaacattgttgtcttggtttgcctaccggtggtaacaattcacctaaaaggacatgcttttagtttgatgggcgcttcaacagtttgcttcttttactctgtgttgttcctaaaactatggtcctatgtgcagacgaatatgtggtgccgtcagacttattatcaaaagaatccgcgggagcgtcgaccaagcataactttggcggaactaaaaaaaggagttttgaatggaggtgaagaagacgaggacgtttccaagctggtgcaatatcctgataatctcacatacaatgatctcctgtacttcctttgcgcgcccactctctgctatgagttgaatttcccgcgaacttctcgcgtgcgcaaacgctttttgctgaagcgtttattggaggtggtgattggagtgaatgtggttatggccttgtttcaacaatggatcattccatcggttcggaactccctgattccgttctccaatatggacgtggccttagccactgagcgacttcttaaacttgcgctacccaatcatctttgctggctctgctttttctatctaatgttccactcttttcttaatgcggtcggcgaactgctgaactttgcagatcgcaatttttattgtgattggtggaatgcgaataacattgacaccttctggcgtacatggaacatgccagttcataggtggtgcgtgcgtcatctctacatccctgtggtccaaatgggatattcctcaagacaggcctctactattgtctttcttttcagtgccgtcttccatgaatatttggtttcagttcctttgcaaatatacaagatctgggcatttatgggcatgatgggtcagattcccctatcggccatatccaaatccattgaaaagaaactgggtccccgaatgggcaatataatcgtgtgggcttccattattcttggtcagcctctgtgcataatggcctattatcacgattatgtcgtccagcatttcaaaaactcgctcaacggcaccgactacagtagttag

SEQ ID NO: 7 - DNA, 1470 bp, Sus scrofa
  CDS (1)...(1470): Sus scrofa diacylglycerol acyltransferase (DGAT) coding sequence
atgggtgaccgcagcggcgcgggcggctcccggcgccggaggacggggtcgcggccctccagccagagcggcagcgggttcgcggccgcagaagaggaggtgcgggacgtaggcgccgggggggacgcaccgacgccggacaaggacaaggacggacacgacgatgtgagcagcggccactgggatctgaggtgccaccgcctgcaggattctttgttcagttcagacagtggtttcagcaactaccgtggcatcctgaattggtgtgtggtcatgctggtcttgagcaatgcacggctgtttctagagaacctcatcaagtacggcatcctggtagaccccatccaggtggtgtctctgttcctgaaggacccctatagctggcctgccctgtgcctggttattgtggccaatgtctttgctgtgactgcgttccaggtggagaagcgcctggccgtgggtgccctgaccgagcaggcggggctgctgatccacgtggccaacctggccaccatcctctgcttcccagcggccgtggctttcctgctggagtccatcactccagtgggctccctgctggctctgatggtctacgccatcctcttcctcaagctgttctcctaccgggacgtcaacctgtggtgccgagagcgcagggctactgccaaggccaaggccgcttctgcaggtaagaaggccaacgggggcgccgcccagcacagcgtgagctaccccgacaacctgacctaccgcgatctctactacttcctcctggccccgactctgtgctacgagctcaacttttcccgcttcccgcgcatccgaaagcgcttcctgctgcggcggctcctggagatgctgttcctcatccagctgcaggtggggctgatccagcagtggatggtccccaccatccagaactccatgaagcccttcaaggacatggactactcacgcatcatcgagcgcctcctgaagctggcggtgcccaaccacctcatctggctcatcttcttctactggctcttccactcctgcctgaacgctgtggctgagctcatgcagtttggagaccgggagttctaccgggactggtggaactcggagtctgtcacctacttctggcagaactggaacatccctgtacacaagtggtgcctcagacacttctacaagcccatgctccggcggggcagcagcaagtgggtagccaggatgggggtgttcctggcttcagccttcttccacgagtacctggtgagcatccctctgcgcatgttccgcctctgggccttcacgggcatgatggctcagatcccgctggcttggatagtgggccgcttcttccgtggcaactacggcaacgcggctgtgtggctgtcgctcatcatcgggcagccggtggccgtgctcatgtacgtccatgactactacgtgctccaccacgaggccccgacagcgggggcctga

SEQ ID NO: 8 - DNA, 1167 bp, Homo sapiens
  CDS (1)...(1167): Homo sapiens DGAT 2 coding sequence
atgaagaccctcatagccgcctactccggggtcctgcgcggcgagcgtcaggccgaggctgaccggagccagcgctctcacggaggacctgcgctgtcgcgcgaggggtctgggagatggggcactggatccagcatcctctccgccctccaggacctcttctctgtcacctggctcaataggtccaaggtggaaaagcagctacaggtcatctcagtgctccagtgggtcctgtccttccttgtactgggagtggcctgcagtgccatcctcatgtacatattctgcactgattgctggctcatcgctgtgctctacttcacttggctggtgtttgactggaacacacccaagaaaggtggcaggaggtcacagtgggtccgaaactgggctgtgtggcgctactttcgagactactttcccatccagctggtgaagacacacaacctgctgaccaccaggaactatatctttggataccacccccatggtatcatgggcctgggtgccttctgcaacttcagcacagaggccacagaagtgagcaagaagttcccaggcatacggccttacctggctacactggcaggcaacttccgaatgcctgtgttgagggagtacctgatgtctggaggtatctgccctgtcagccgggacaccatagactatttgctttcaaagaatgggagtggcaatgctatcatcatcgtggtcgggggtgcggctgagtctctgagctccatgcctggcaagaatgcagtcaccctgcggaaccgcaagggctttgtgaaactggccctgcgtcatggagctgacctggttcccatctactcctttggagagaatgaagtgtacaagcaggtgatcttcgaggagggctcctggggccgatgggtccagaagaagttccagaaatacattggtttcgccccatgcatcttccatggtcgaggcctcttctcctccgacacctgggggctggtgccctactccaagcccatcaccactgttgtgggagagcccatcaccatccccaagctggagcacccaacccagcaagacatcgacctgtaccacaccatgtacatggaggccctggtgaagctcttcgacaagcacaagaccaagttcggcctcccggagactgaggtcctggaggtgaactga

SEQ ID NO: 9 - DNA, 1467 bp, Homo sapiens
  CDS (1)...(1467): Homo sapiens diacylglycerol O-acyltransferase homolog 1 (DGAT1), coding sequence
atgggcgaccgcggcagctcccggcgccggaggacagggtcgcggccctcgagccacggcggcggcgggcctgcggcggcggaagaagaggtgcgggacgccgctgcgggccccgacgtgggagccgcgggggacgcgccagccccggcccccaacaaggacggagacgccggcgtgggcagcggccactgggagctgaggtgccatcgcctgcaggattctttattcagctctgacagtggcttcagcaactaccgtggcatcctgaactggtgtgtggtgatgctgatcttgagcaatgcccggttatttctggagaacctcatcaagtatggcatcctggtggaccccatccaggtggtttctctgttcctgaaggatccccatagctggcccgccccatgcctggttattgcggccaatgtctttgctgtggctgcattccaggttgagaagcgcctggcggtgggtgccctgacggagcaggcgggactgctgctgcacgtagccaacctggccaccattctgtgtttcccagcggctgtggtcttactggttgagtctatcactccagtgggctccctgctggcgctgatggcgcacaccatcctcttcctcaagctcttctcctaccgcgacgtcaactcatggtgccgcagggccagggccaaggctgcctctgcagggaagaaggccagcagtgctgctgccccgcacaccgtgagctacccggacaatctgacctaccgcgatctctactacttcctcttcgcccccaccttgtgctacgagctcaactttccccgctctccccgcatccggaagcgctttctgctgcgacggatccttgagatgctgttcttcacccagctccaggtggggctgatccagcagtggatggtccccaccatccagaactccatgaagcccttcaaggacatggactactcacgcatcatcgagcgcctcctgaagctggcggtccccaatcacctcatctggctcatcttcttctactggctcttccactcctgcctgaatgccgtggctgagctcatgcagtttggagaccgggagttctaccgggactggtggaactccgagtctgtcacctacttctggcagaactggaacatccctgtgcacaagtggtgcatcagacacttctacaagcccatgcttcgacggggcagcagcaagtggatggccaggacaggggtgttcctggcctcggctttcttccacgagtacctggtgagcgtccctctgcgaatgttccgcctctgggctttcacgggcatgatggctcagatcccactggcctggttcgtgggccgctttttccagggcaactatggcaacgcagctgtgtggctgtcgctcatcatcggacagccaatagccgtcctcatgtacgtccacgactactacgtgctcaactatgaggccccagcggcagaggcctga

SEQ ID NO: 10 - DNA, 1005 bp, Homo sapiens
  CDS (1)...(1005): Homo sapiens diacylglycerol acyltransferase 2-like protein coding sequence
atgaaggtagagtttgcaccgctcaacatccagctggcgcggcggctgcagacggtggccgtgctgcagtgggtcctttcttttcttacagggccgatgtccattggaatcactgtgatgctgatcatacacaactatttgttcctttacatcccttatttgatgtggctttactttgactggcataccccagagcgaggaggcaggagatccagctggatcaaaaattggactctttggaaacactttaaggactattttccaattcatcttatcaaaactcaagatttggatccaagtcacaactatatatttgggtttcacccccatggaataatggcagttggagcctttgggaatttttctgtaaattattctgacttcaaggacctgtttcctggctttacttcatatcttcacgtgctgccactttggttctggtgtcctgtctttcgagaatatgtgatgagtgttgggctggtttcagtttccaagaaaagtgtgtcctacatggtaagcaaggagggaggtggaaacatctctgtcattgtccttgggggtgcaaaagaatcactggatgctcatcctggaaagttcactctgttcatccgccagcggaaaggatttgttaaaattgctttgacccatggcgcctctctggtcccagtggtttcttttggtgaaaatgaactgtttaaacaaactgacaaccctgaaggatcatggattagaactgttcagaataaactgcagaagatcatggggtttgctttgcccctgtttcatgccaggggagtttttcagtacaattttggcctaatgacctataggaaagccatccacactgttgttggccgcccgatccctgttcgtcagactctgaacccgacccaggagcagattgaggagttacatcagacctatatggaggaacttaggaaattgtttgaggaacacaaaggaaagtatggcattccagagcacgagactcttgttttaaaatga

SEQ ID NO: 11 - DNA, 1008 bp, Mus musculus
  CDS (1)...(1008): Mus musculus diacylglycerol acyltransferase 2-like protein coding sequence
atgatggtcgagttcgcgccactcaacaccccgctggcacggtgcctacagaccgctgcggtgctgcagtgggtcctgtccttcctcctgctcgtgcaggtgtgcattggaattatggtgatgctggtcctgtacaactattggttcctttacatcccatatctggtctggttttactatgactggagaaccccagagcaaggaggcagaagatggaactgggtccaaagctggcctgtgtggaagtattttaaggagtattttccaatctgtcttgtcaaaacgcaggatttggatccgggtcacaattatatatttgggtttcaccctcatggaatattcgtgcctggagcctttggaaatttttgtacaaaatactcggacttcaagaagctatttcctggctttacatcgtatctccacgtggccaagatctggttctgtttcccgttgttccgagaatatctgatgagtaacgggccggtttcagtgtctaaggagagtttgtctcatgtgctgagcaaggatggaggtggcaatgtctcaatcattgtcctcggaggtgcaaaggaggcgctggaggctcacccaggaacattcaccctgtgcatccgccagcgcaaagggtttgttaagatggccttgacccatggtgccagtttggttccagtattttcttttggtgaaaatgatctatataagcaaattaacaaccccaaaggctcctggctacgaactatacaagacgcaatgtatgattcaatgggagtagccttgccactgatatatgccagaggaattttccagcactactttggcataatgccctatcggaagctgatctacactgttgttggccgccctatccctgttcagcagattctgaacccgacctcagagcagattgaagagctgcatcagacatacctagaggagctaaagaaactattcaatgaacacaaagggaaatatgggattccggagcacgaaactctggtatttaaataa

SEQ ID NO: 12 - DNA, 1497 bp, Mus musculus; identical to SEQ ID NO: 1
  CDS (1)...(1497): Mus musculus diacylglycerol acyltransferase (Dgat) coding sequence

SEQ ID NO: 13 - DNA, 504 bp, Rattus norvegicus
  CDS (1)...(54): Rattus norvegicus leptin (Lep), coding sequence
atgtgctggagacccctgtgccggttcctgtggctttggtcctatctgtcctatgttcaagctgtgcctatccacaaagtccaggatgacaccaaaaccctcatcaagaccattgtcaccaggatcaatgacatttcacacacgcagtcggtatccgccaggcagagggtcaccggtttggacttcattcccgggcttcaccccattctgagtttgtccaagatggaccagaccctggcagtctatcaacagatcctcaccagcttgccttcccaaaacgtgctgcagatagctcatgacctggagaacctgcgagacctcctccatctgctggccttctccaagagctgctccctgccgcagacccgtggcctgcagaagccagagagcctggatggcgtcctggaagcctcgctctactccacagaggtggtggctctgagcaggctgcagggctctctgcaggacattcttcaacagttggaccttagccctgaatgctga

SEQ ID NO: 14 - DNA, 2679 bp, Mus musculus
  CDS (1)...(2679): Mus musculus leptin receptor (Lepr) coding sequence
atgatgtgtcagaaattctatgtggttttgttacactgggaatttctttatgtgatagctgcacttaacctggcatatccaatctctccctggaaatttaagttgttttgtggaccaccgaacacaaccgatgactcctttctctcacctgctggagccccaaacaatgcctcggctttgaagggggcttctgaagcaattgttgaagctaaatttaattcaagtggtatctacgttcctgagttatccaaaacagtcttccactgttgctttgggaatgagcaaggtcaaaactgctctgcactcacagacaacactgaagggaagacactggcttcagtagtgaaggcttcagtttttcgccagctaggtgtaaactgggacatagagtgctggatgaaaggggacttgacattattcatctgtcatatggagccattacctaagaaccccttcaagaattatgactctaaggtccatcttttatatgatctgcctgaagtcatagatgattcgcctctgcccccactgaaagacagctttcagactgtccaatgcaactgcagtcttcggggttgtgaatgtcatgtgccggtacccagagccaaactcaactacgctcttctgatgtatttggaaatcacatctgccggtgtgagttttcagtcacctctgatgtcactgcagcccatgcttgttgtgaaacccgatccacccttaggtttgcatatggaagtcacagatgatggtaatttaaagatttcttgggacagccaaacaatggcaccatttccgcttcaatatcaggtgaaatatttagagaattctacaattgtaagagaggctgctgaaattgtctcagctacatctctgctggtagacagtgtgcttcctggatcttcatatgaggtccaggtgaggagcaagagactggatggttcaggagtctggagtgactggagttcacctcaagtctttaccacacaagatgttgtgtattttccacccaaaattctgactagtgttggatcgaatgcttcttttcattgcatctacaaaaacgaaaaccagattatctcctcaaaacagatagtttggtggaggaatctagctgagaaaatccctgagatacagtacagcattgtgagtgaccgagttagcaaagttaccttctccaacctgaaagccaccagacctcgagggaagtttacctatgacgcagtgtactgctgcaatgagcaggcgtgccatcaccgctatgctgaattatacgtgatcgatgtcaatatcaatatatcatgtgaaactgacgggtacttaactaaaatgacttgcagatggtcacccagcacaatccaatcactagtgggaagcactgtgcagctgaggtatcacaggcgcagcctgtattgtcctgatagtccatctattcatcctacgtctgagcccaaaaactgcgtcttacagagagacggcttttatgaatgtgttttccagccaatctttctattatctggctatacaatgtggatcaggatcaaccattctttaggttcacttgactcgccaccaacgtgtgtccttcctgactccgtagtaaaaccactacctccatctaacgtaaaagcagagattactgtaaacactggattattgaaagtatcttgggaaaagccagtctttccggagaataaccttcaattccagattcgatatggcttaagtggaaaagaaatacaatggaagacacatgaggtattcgatgcaaagtcaaagtctgccagcctgctggtgtcagacctctgtgcagtctatgtggtccaggttcgctgccggcggttggatggactaggatattggagtaattggagcagtccagcctatacgcttgtcatggatgtaaaagttcctatgagagggcctgaattttggagaaaaatggatggggacgttactaaaaaggagagaaatgtcaccttgctttggaagcccctgacgaaaaatgactcactgtgtagtgtgaggaggtacgtggtgaagcatcgtactgcccacaatgggacgtggtcagaagatgtgggaaatcggaccaatctcactttcctgtggacagaaccagcgcacactgttacagttctggctgtcaattccctcggcgcttcccttgtgaattttaaccttaccttctcatggcccatgagtaaagtgagtgctgtggagtcactcagtgcttatcccctgagcagcagctgtgtcatcctttcctggacactgtcacctgatgattatagtctgttatatctggttattgaatggaagatccttaatgaagatgatggaatgaagtggcttagaattccctcgaatgttaaaaagttttatatccacgataattttattcccatcgagaaatatcagtttagtctttacccagtatttatggaaggagttggaaaaccaaagataattaatggtttcaccaaagatgctatcgacaagcagcagaatgacgcagggctgtatgtcattgtacccataattatttcctcttgtgtcctactgctcggaacactgttaatttcacaccagagaatgaaaaagttgttttgggacgatgttccaaaccccaagaattgttcctgggcacaaggactgaatttccaaaaggtcactgtttaa

SEQ ID NO: 15 - DNA, 504 bp, Mus musculus
  CDS (1)...(504): Mus musculus leptin (Lep)
atgtgctggagacccctgtgtcggttcctgtggctttggtcctatctgtcttatgttcaagcagtgcctatccagaaagtccaggatgacaccaaaaccctcatcaagaccattgtcaccaggatcaatgacatttcacacacgcagtcggtatccgccaagcagagggtcactggcttggacttcattcctgggcttcaccccattctgagtttgtccaagatggaccagactctggcagtctatcaacaggtcctcaccagcctgccttcccaaaatgtgctgcagatagccaatgacctggagaatctccgagacctcctccatctgctggccttctccaagagctgctccctgcctcagaccagtggcctgcagaagccagagagcctggatggcgtcctggaagcctcactctactccacagaggtggtggctttgagcaggctgcagggctctctgcaggacattcttcaacagttggatgttagccctgaatgctga

SEQ ID NO: 16 - DNA, 363 bp, Bos taurus
  unsure (0)...(0): Bos taurus leptin receptor partial coding sequence
ctggaaatttaagttgtcttgcatgccatcaaatacaacatatgacttcctctttcctcctggaatctcaaagaacacttcaaatttgaatggacgttatgaggcagttgttgaaactaagcttaattcaagtggtacctacttatctaacttatcatccagaacaactttccactgttgcttttggagcgcggaagataaaaactgctctgtatatacagatgacattgaagggaaggcatttgttacaacagtaaattctttagtttttcagcaaacaggtgcaaactggaacatacagtgctggatgaaagaggacttgaaattattcatctgtaatattgagtcattatttaagaatcc

SEQ ID NO: 17 - DNA, 504 bp, Homo sapiens
  CDS (1)...(504): Homo sapiens leptin
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctga

SEQ ID NO: 18 - DNA, 3489 bp, Rattus norvegicus
  CDS (1)...(3489): Rattus norvegicus leptin receptor
atgacgtgtcagaaattctatgtggttttgttacactgggaatttctgtatgtgataactgcacttaacctggcctatccaacctctccctggagatttaagctgttttgtgcgccaccgagtacaactgatgactcctttctctctcctgctggagtcccaaacaatacttcgtctttgaagggggcttctgaagcacttgttgaagctaaatttaattcaactggtatctacgtttctgagttatccaaaaccattttccactgttgctttgggaatgagcaaggtcaaaactgctccgcactcacaggcaacactgaagggaagacgctggcttcagtggtgaagcctttagttttccgccaactaggtgtaaactgggacatagagtgctggatgaaaggggacttgacattattcatctgtcatatggaaccattacttaagaaccccttcaagaattatgactctaaggttcaccttttatatgatctgcctgaagttatagatgatttgcctctgcccccactgaaagacagctttcagactgtccagtgcaactgcagtgttcgggaatgcgaatgtcatgtaccagtacccagagccaaagtcaactacgctcttctgatgtatttagaaatcacatctgctggtgtgagttttcagtcacctctaatgtcactgcagcccatgcttgttgtgaagcccgatccaccgctgggtttgcgtatggaagtcacagatgatggtaatttaaagatttcatgggacagccaaacaaaagcaccatttccacttcaatatcaggtgaaatatttagagaattctacaatcgtaagagaggctgctgaaatcgtctcggatacatctctgctggtagacagcgtgcttcctgggtcttcatacgaggtccaggtgaggagcaagagactggatggctcaggagtctggagtgactggagtttacctcaactctttaccacacaagatgtcatgtattttccacccaaaattctgacgagtgttggatccaatgcttccttttgctgcatctacaaaaatgagaaccagactatctcctcaaaacaaatagtttggtggatgaatctagccgagaagatccccgagacacagtacaacactgtgagtgaccacattagcaaagtcactttctccaacctgaaagccaccagacctcgagggaagtttacctatgatgcagtgtactgctgcaatgagcaggcatgccatcaccgctacgctgaattatatgtgatcgatgtcaatatcaatatatcatgtgaaactgacgggtacttaactaaaatgacttgcagatggtcacccagcacaatccaatcactagtgggaagcactgtgcagttgaggtatcacaggcgcagcctgtactgtcccgataatccatctattcgtcctacatcagagctcaaaaactgcgtcttacagacagatggcttttatgaatgtgttttccagccaatctttctattatctggctatacaatgtggatcaggatcaaccattctttaggttcacttgactctccaccaacgtgtgtccttcctgactccgtagtaaaaccactacctccatctaatgtaaaagcagagattactataaacactggattattgaaagtatcttgggaaaagccagtctttccagagaataaccttcagttccagattcgatatggcttaaatggaaaagaaatacaatggaagacacacgaggtattcgatgcaaaatcaaaatcggccagcctgccagtgtcagatctctgtgcggtctatgtggtacaggttcgctgccggcggttggatggactagggtattggagtaattggagcagtccagcctacactcttgtcatggatgtaaaagttcctatgagagggcctgaattctggagaataatggatggggatattactaaaaaggagagaaatgtcaccttgctttggaagccactgatgaaaaatgactcactgtgtagtgtgaggaggtatgtggtgaagcatcgtactgcccacaatgggacatggtcacaagatgtgggaaatcagaccaatctcactttcctgtgggcagaatcagcacacactgttacagttctggccatcaattccatcggtgcctcccttgtgaattttaaccttacgttctcatggcccatgagtaaagtgaatgctgtgcagtcactcagtgcttatcccctgagcagcagctgcgtcatcctttcctggacactgtcacctaatgattatagtctgttatatctggttattgaatggaagaaccttaatgatgatgatggaatgaagtggcttagaatcccttcgaatgttaacaagtattatatccatgataattttattcctatcgagaaatatcagtttagtctttacccagtatttatggaaggagttggaaaaccaaagataattaatggtttcaccaaagatgatatcgccaaacagcaaaatgatgcagggctgtatgtcattgtaccgataattatttcctcttgtgtcctgctgctcggaacactgttaatttcacaccagagaatgaaaaagttgttttgggacgatgttccaaaccccaagaattgttcctgggcacaaggacttaatttccaaaagcctgaaacatttgagcatctttttaccaagcatgcagaatcagtgatatttggtcctcttcttctggagcctgaaccagtttcagaagaaatcagtgtcgatacagcttggaaaaataaagatgagatggtaccagcagctatggtctcacttcttttgaccactccagattccacaaggggttctatttgtatcagtgaccagtgtaacagtgctaacttctctggggctcagagcacccagggaacctgtgaggatgagtgtcagagtcaaccctcagttaaatatgcaacgctggtcagcaacgtgaaaacagtggaaactgatgaagagcaaggggctatacatagttctgtcagccagtgcatcgccaggaaacattccccactgagacagtctttttctagcaactcctgggagatagaggcccaggcatttttccttttatcagatcatccacccaatgtgatttcaccacaactttcattctcagggttggatgagcttttggaactggagggaaattttcctgaagaaaatcacggggaaaaatctgtgtattatctaggagtctcctcaggaaacaaaagagagaatgatatgcttttgactgatgaggcaggggtattgtgcccattcccagctcactgtctgttcagtgacatcagaatcctccaggagagttgttcacactttgtagaaaataatttgaatttagggacctctggtaagaactttgtaccttacatgccccagtttcaatcctgttccactcacagtcataagataatagaaaataagatgtgtgacttaactgtgtaa

SEQ ID NO: 19 - DNA, 20 bp, Artificial Sequence
  Synthetic oligonucleotide primer
catcttggcctcactgtcca

SEQ ID NO: 20 - DNA, 19 bp, Artificial Sequence
  Synthetic oligonucleotide primer
gggccggactcatcgtact

SEQ ID NO: 21 - DNA, 26 bp, Artificial Sequence
  Synthetic oligonucleotide primer
cttccagcagatgtggatcagcaagc

SEQ ID NO: 22 - DNA, 23 bp, Artificial Sequence
  Synthetic oligonucleotide primer
agtggcaatgctatcatcatcgt

SEQ ID NO: 23 - DNA, 25 bp, Artificial Sequence
  Synthetic oligonucleotide primer
aaggaataagtgggaacccagatca

SEQ ID NO: 24 - DNA, 19 bp, Artificial Sequence
  Synthetic oligonucleotide primer
caagaacgcagtcaccctg

(19) United States
(12) Patent Application Publication
(10) Pub. No.: US 2003/0100480 A1
(43) Pub. Date: May 29, 2003

(54) Methods and compositions for modulating sebaceous glands

(76) Inventors: Steven J. Smith, San Francisco, CA (US); Hubert C. Chen, San Francisco, CA (US); Robert V. Farese JR., San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/278,733

(22) Filed: Oct. 21, 2002

Related U.S. Application Data
(63) Continuation-in-part of application No. 10/040,315, filed on Oct. 29, 2001.
Application No. 10/040,315 is a continuation-in-part of application No. 09/339,472, filed on Jun. 23, 1999, now abandoned.
Application No. 10/040,315 is a continuation-in-part of application No. PCT/US98/17883, filed on Aug. 28, 1998.
Application No. PCT/US98/17883 is a continuation-in-part of application No. 09/103,754, filed on Jun. 24, 1998, now Pat. No. 6,344,548.
(60) Provisional application No. 60/107,771, filed on Nov. 9, 1998.

(51) Int. Cl. A61K 31/00; A61K 48/00

(52) U.S. Cl. 514/1; 514/44

(57) ABSTRACT

Methods and compositions for modulating sebaceous gland activity in a host are provided. In the subject methods, DGAT1 activity is modified, e.g., reduced or enhanced, to achieve the desired sebaceous gland activity modulation, e.g., reduction in sebum production and/or sebaceous gland size. Also provided are pharmaceutical preparations for use in practicing the subject methods. The subject methods and compositions find use in a variety of applications, including the treatment of hosts suffering from sebaceous gland related conditions, e.g., acne and related conditions.

### CROSS REFERENCE TO RELATED APPLICATIONS

This application is a continuation-in-part of application Ser. No. 10/040,315 filed Oct. 29, 2001; which application is: (a) a continuation-in-part of application Ser. No. 09/339,472 filed on Jun. 23, 1999, which application claims priority to the filing date of U.S. Provisional Patent Application Serial No. 60/107,771 filed Nov. 9, 1998; and (b) a continuation-in-part of PCT application Ser. No. PCT/US98/17883, filed Aug. 28, 1998, which application is a continuation in part of application Ser. No. 09/103,754, now U.S. Pat. No. 6,344,548, filed Jun. 24, 1998; the disclosures of which applications are herein incorporated by reference.

### ACKNOWLEDGMENT OF GOVERNMENT SUPPORT

This invention was made with Government support under Grant Nos. R01 52069; R01 57170 and R01 DK56084 awarded by the National Institutes of Health. The Government has certain rights in this invention.

### INTRODUCTION

1. Field of the Invention

The invention relates generally to methods of treating a mammalian skin disorder associated with sebaceous glands.

2. Background of the Invention

Sebaceous glands are associated with hair follicles and produce an oil called sebum, which is secreted into the hair follicle to keep the skin hair supple and waterproof. There are a variety of disorders associated with altered activity of sebaceous glands. Such disorders include acne, rosacea, perioral dermatitis, sebaceous cysts and seborrhea (greasy complexion or hair) and alopecia (baldness). In humans, sebaceous glands, although present over most of the body surface, usually are largest and most dense on the face, chest and upper back. Accordingly, sebaceous gland disorders predominantly affect these areas of the human body.

The most pervasive sebaceous follicle disorder in the United States is acne. Acne is characterized by inflammatory, follicular, papular and/or pustular eruptions involving sebaceous glands (Stedman's Medical Dictionary, 26th edition, (1995) Williams & Wilkins) and affects between 40 to 50 million individuals (White G M, (1998) J. Am. Acad. Dermatol. 39(2 Pt 3): S34-7). Although there are a variety of disorders that fall within the acne family, for example, acne conglobata, acne rosacea, and acne vulgaris, acne vulgaris is the most notable and commonly known form of acne. Acne vulgaris occurs with greatest frequency in individuals between the ages of 15 and 18 years, but may begin at virtually any age and can persist into adulthood. Because acne vulgaris can lead to permanent scarring, for example, facial scarring, this form of acne can have profound and long-lasting psychological effects on an afflicted individual. Furthermore, pustule formation and scarring can occur at an age when the potential impact on an individual is greatest, e.g., during adolescence.

Acne vulgaris typically results from a blockage of the opening of the sebaceous follicle. It is believed that both (i) the amount of sebum, (a lipid, keratin and cellular debris-containing fluid), produced and secreted by the sebaceous glands and (ii) bacteria, namely, Propionibacterium acnes (P. acnes) which metabolize lipids in the sebum, play a role in formation and development of acne vulgaris. The basic lesion of acne vulgaris is referred to as a comedo, a distension of the sebaceous follicle caused by sebum and keratinous debris. Formation of a comedo usually begins with defective keratinization of the follicular duct, resulting in abnormally adherent epithelial cells and plugging of the duct. When sebum production continues unabated, the plugged follicular duct distends. A blackhead (or open comedo) occurs when a plug comprising a melanin containing blackened mass of epithelial debris pushes up to opening of the follicular duct at the skin surface. A whitehead (or closed comedo) occurs when the follicle opening becomes very tightly closed and the material behind the closure ruptures the follicle causing a low-grade dermal inflammatory reaction. Accordingly, some comedos, for example, in acne vulgaris, evolve into inflammatory papules, pustules, nodules, or chronic granulomatous lesions. Proliferation of P. acnes can result in the production of inflammatory compounds, eventually resulting in neutrophil chemotaxis (Skyes and Webster (1994) Drugs 48: 59-70).

Typical short term treatments for sebaceous gland disorders include numerous cleansing methods that attempt to relieve the symptoms of the disorder. These treatments include special soaps, skin-peeling compositions, shampoos and the like, that may be used to, for example, remove comedos, prevent comedo formation and reduce the greasiness of hair. Many of the preparations attempt to reduce the tendency for acne by using drying, keratolytic, and antibacterial active ingredients. Skin cleansing degreases and extracts moisture from the skin and has the disadvantage that the water-insoluble calcium and magnesium salts of higher fatty acids, which form when the soaps are used in hard water, form slimy precipitates on the skin. Because they are difficult to rinse off, these precipitates remain for a relatively long period on the skin, block the follicle openings and can lead to the formation of more sebaceous gland problems. Syndets, i.e. surfactants without soap character, have been used to attempt to solve this problem, however, the use of most cleansing agents containing syndets often leads to a reduction in the water content in the upper layers of the skin, which in turn can induce inflammation.

For long-term treatment of sebaceous gland disorders, many acne patients may receive years of chronic topical or systemic treatments. Current treatment options include, for example, the use of topical anti-inflammatory agents, antibiotics and peeling agents, oral antibiotics, topical and oral retinoids, and hormonal agonists and antagonists. Topical agents include, for example, retinoic acid, benzoyl peroxide, and salicylic acid (Harrison's Principles of Internal Medicine, 14.sup.th edition, (1998) Fauci et al., eds. McGraw-Hill). Useful topical antibiotics include, for example, clindamycin, erythromycin, and tetracycline and useful systemic antibiotics include, for example, erythromycin, tetracycline, and sulphanilamides (see, for example, U.S. Pat. Nos. 5,910,493 and 5,674,539). Administration of the systemic retinoid, isotretinion, has demonstrated some success in the treatment of acne (Harrison's Principles of Internal Medicine, 14.sup.th edition, (1998) Fauci et al., eds. McGraw-Hill). Studies indicate that this drug decreases sebaceous gland size, decreases the rate of sebum production and/or secretion, and causes ductal epithelial cells to be less adherent, thereby preventing precursor lesions of acne vulgaris (Skyes and Webster (1994) supra). Side-effects, however, include dry mouth and skin, itching, small red spots in the skin, and eye irritation. A significant concern about oral retinoids is their possible teratogenicity (Turkington and Dover (1996) Skin Deep: An A-Z Of Skin Disorders, Treatment And Health Facts On File, Inc., New York, page 9). In addition, a variety of hormone-related therapies have been developed for the treatment of acne. These therapies can be expensive and most are associated with deleterious systemic or localized side-effects (Strauss (1982) Curr. Med. Res. Opin. 7(Suppl 2): 33-45).

As such, there is still not an optimal method for treating acne or other sebaceous gland disorders. Accordingly, there is an ongoing need for methods of effectively treating sebaceous gland disorders, particularly acne and seborrhea. This invention meets this, and other needs.

Relevant Literature

U.S. Patents and published patent applications of interest include: U.S. Pat. Nos. 6,100,077 and 6,344,548. Also of interest are Chen et al., J. Clin. Invest. (January, 2002) 109:175-181; Cases et al., Proc. Nat'l Acad. Sci. USA (1998) 95:13018-13023 and Cases et al., J. Biol. Chem. (2001) 276:38870-38876.

### SUMMARY OF THE INVENTION

Methods and compositions for modulating sebaceous gland activity in a host are provided. In the subject methods, DGAT1 activity is modulated, e.g., inhibited or enhanced, to achieve the desired sebaceous gland modulation, e.g., reduction in sebum production and/or sebaceous gland size. Also provided are pharmaceutical preparations for use in practicing the subject methods. The subject methods and compositions find use in a variety of applications, including the treatment of hosts suffering from such conditions.

### BRIEF DESCRIPTION OF THE FIGURES

FIGS. 1a to 1d show photographs (FIGS. 1a and b) and line graphs (FIGS. 1c and d) showing the effects of DGAT1 deficiency on fur appearance, water repulsion, and thermoregulation in mice. FIG. 1a: Dry fur and hair loss in a 16-week-old male Dgat^{-/-} mouse. FIG. 1b: Male Dgat^{+/+} and Dgat^{-/-} mice 5 minutes after water immersion. FIGS. 1c and d: Impaired water repulsion and thermoregulation in Dgat^{-/-} mice after water immersion. Dgat^{-/-} mice retained more water in their fur than did Dgat^{+/+} mice, as reflected by a greater increase in mean relative body weight (FIG. 1 panel c). Dgat^{-/-} mice also developed hypothermia (FIG. 1d). n=4 per genotype. *P<0.05.

FIGS. 2a to 2h provide a series of line graphs showing abnormalities of water repulsion and thermoregulation in DGAT1-deficient AY/a but not ob/ob mice. FIGS. 2a and b: Effect of DGAT1 deficiency on water repulsion (FIG. 2a) and thermoregulation (FIG. 2b) of AY/a mice after water immersion. FIGS. 2c-h: Effect of DGAT1 deficiency on water repulsion and thermoregulation of ob/ob mice after water immersion. FIGS. 2c and d: No leptin infusion. FIGS. 2e and f: After 2 weeks of subcutaneous leptin infusion (+Peripheral leptin). FIGS. 2g and h: Two weeks after the leptin infusion was stopped (After leptin). For each experiment, n=4 per genotype. *P<0.05.

FIGS. 3a and 3b are two photographs showing DGAT mRNA expression in skin. An antisense probe detected DGAT1 mRNA expression in sebaceous glands (arrows, FIG. 3A) of skin from wild-type mice. Specific hybridization was not detected by the control sense probe (FIG. 3B).

FIGS. 4a-4d are a series of photographs showing that age modulates the effect of DGAT1 deficiency on sebaceous gland morphology. (FIGS. 4a and b) In 6-week-old male mice, the sebaceous glands (SG) and hair follicles (HF) appeared to be normal, regardless of Dgat genotype. (FIGS. 4c and d) In 3-month-old male mice, DGAT1 deficiency was associated with atrophic sebaceous glands; for most hair follicles, sebaceous glands were not identifiable. Bar is 30 μm.

FIGS. 5a-5j are a series of photographs showing sebaceous gland abnormalities in DGAT1-deficient AY/a but not ob/ob mice. FIGS. 5a and b: Skin section from Dgat^{+/+} AY/a mice (FIG. 5a) and Dgat^{-/-} AY/a mice (FIG. 5b). FIGS. 5c-j: skin sections from Dgat^{+/+} ob/ob and Dgat^{-/-} ob/ob mice. FIGS. 5c and d: No leptin infusion. FIGS. 5e and f: After 2 weeks of subcutaneous leptin infusion (+Peripheral leptin). FIGS. 5g and h: After 2 weeks of intracerebroventricular leptin infusion (+Central leptin). FIGS. 5i and j: Two weeks after the leptin infusion was stopped (After leptin). Representative samples from male mice are shown. SG, sebaceous gland. Bar, 30 μm.

FIGS. 6a and 6b are two photographs showing abnormal fur lipid content in Dgat^{-/-} mice. FIG. 6a: Absence of specific lipids in the fur of Dgat^{-/-} mice (white and gray arrows). FIG. 6b: Effects of leptin on fur lipid content. The putative type II wax diester is indicated with a white arrow. Lipids were analyzed by TLC with hexane/ethyl ether/acetic acid (FIG. 6a) and hexane/benzene (FIG. 6b). Experiments were performed 3-4 times. Representative results are shown.

FIG. 7 is a photograph showing affects of androgens on fur lipids in Dgat^{-/-} mice. The putative type II wax diester is indicated with an open arrow. Lipids were analyzed by TLC with hexane/benzene. For lanes 3 and 4, testosterone propionate was injected subcutaneously for 2 weeks. For lanes 5 and 6, fur lipids were extracted 2 weeks after castration. Experiments were performed twice. Representative results are shown.

FIGS. 8a and 8b are two bar graphs showing upregulation of DGAT2 in the skin of ob/ob mice. FIG. 8a: mRNA expression of DGAT2. n=3 per genotype. *P<0.05 vs. Dgat^{+/+}. FIG. 8b: Increased upregulation of DGAT2 mRNA expression in ob/ob mice with DGAT1 deficiency. n=3 per genotype. *P<0.05.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

As used herein, the terms “sebaceous gland disorder” or “sebaceous gland condition” are used interchangeably to refer to any disorder that is caused by an alteration in the function of a sebaceous gland. Sebaceous gland disorders may be caused by overactive sebaceous glands, underactive sebaceous glands, mal-developed sebaceous glands, blocked sebaceous glands, infected sebaceous glands, inflamed sebaceous glands and the like. Examples of sebaceous gland disorders include, but are not limited to: acne, including open comedos (blackheads) and whiteheads, pimples, deep acne, acne conglobata, acne rosacea, comedos, cysts, microcomedos, papules, Propionibacterium acnes (P. acnes) infections, pustules, acne vulgaris, rosacea, perioral dermatitis, sebaceous cysts, primary seborrhea (seborrhea oleosa), secondary seborrhea (seborrhea sicca) and alopecia. Also within this definition are disorders treatable by altering the function of a sebaceous gland, such as dandruff and dry skin, and “cosmetic” sebaceous gland disorders, including dry hair, greasy hair, hair and skin sheen and other minor cosmetic disorders of the skin and/or complexion.

The term “phenomenon associated with sebaceous glands” as used herein refers to a structural, molecular, or functional characteristic associated with sebaceous gland function, particularly such a characteristic that is readily assessable in an animal model. Such characteristics include, but are not limited to, DGAT expression and/or activity, lipid production and/or secretion, water repulsion, hair sheen, thermoregulation, hair drying and the like.

As used herein, the terms “determining,” “measuring,” and “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “polynucleotide” and “nucleic acid molecule” are used interchangeably herein to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, double-stranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially pure” refers to a compound that is removed from its natural environment and is at least 60% free, preferably 75% free, and most preferably 90% free from other components with which it is naturally associated.

By “transgenic animal” is meant a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal according to methods well known in the art. A “transgene” is meant to refer to such heterologous nucleic acid, e.g., heterologous nucleic acid in the form of an expression construct (e.g., for the production of a “knock-in” transgenic animal) or a heterologous nucleic acid that upon insertion within or adjacent a target gene results in a decrease in target gene expression (e.g., for production of a “knock-out” transgenic animal).

A “knock-out” of a gene means an alteration in the sequence of the gene that results in a decrease of function of the target gene, preferably such that target gene expression is undetectable or insignificant. Transgenic knock-out animals can be comprise a heterozygous knock-out of a target gene, or a homozygous knock-out of a target gene. “Knock-outs” as used herein also include conditional knock-outs, where alteration of the target gene can occur upon, for example, exposure of the animal to a substance that promotes target gene alteration, introduction of an enzyme that promotes recombination at the target gene site (e.g., Cre in the Cre-lox system), or other method for directing the target gene alteration postnatally.

A “knock-in” of a target gene means an alteration in a host cell genome that results in altered expression (e.g., increased (including ectopic) or decreased expression) of a target gene, e.g., by introduction of an additional copy of the target gene, or by operatively inserting a regulatory sequence that provides for enhanced expression of an endogenous copy of the target gene. “Knock-in” transgenics can comprise a heterozygous knock-in of the target gene or a homozygous knock-in of a target gene. “Knock-ins” also encompass conditional knock-ins.

By “operably linked” is meant that a DNA sequence and a regulatory sequence(s) are connected in such a way as to permit gene expression when the appropriate molecules (e.g., transcriptional activator proteins) are bound to the regulatory sequence(s).

By “operatively inserted” is meant that a nucleotide sequence of interest is positioned adjacent a nucleotide sequence that directs transcription and translation of the introduced nucleotide sequence of interest.

The term “therapeutic agent” as used herein refers to any molecule, e.g., protein or small molecule, pharmaceutical compound, antibody, antisense molecule, ribozyme, and the like, useful in the treatment of a disease or condition, e.g., a sebaceous gland condition. For example, therapeutic agents of the invention include molecules that inhibit, ameliorate, or relieve symptoms associated with a sebaceous gland condition.

The term “unit dosage form” as used herein refers to physically discrete units suitable as unitary dosages for subjects (e.g., animals, usually humans), each unit containing a predetermined quantity of agent(s) in an amount sufficient to produce the desired effect in association with a pharmaceutically acceptable diluent, carrier or vehicle. The specifications for the novel unit dosage forms of the present invention will depend on a variety of factors including, but not necessarily limited to, the particular agent employed and the effect to be achieved, and the pharmacodynamics associated with each compound in the host.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes: (a) preventing the disease from occurring in a subject which may be predisposed to the disease but has not yet been diagnosed as having it; (b) inhibiting the disease; i.e., arresting its development; or (c) relieving the disease, i.e., causing regression of the disease.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

### DETAILED DESCRIPTION OF THE INVENTION

Methods and compositions for modulating sebaceous gland activity in a host are provided. In the subject methods, DGAT1 activity is modulated, e.g., inhibited or enhanced, to achieve the desired sebaceous gland modulation, e.g., reduction in sebum production and/or sebaceous gland size. Also provided are compositions for use in practicing the subject methods. The subject methods and compositions find use in a variety of applications, including the treatment of hosts suffering from conditions.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

In further describing the invention, representative methods of modulating sebaceous glands in a host (as well as compositions for use in such methods) are reviewed first followed by a more detailed description of representative applications in which the subject methods find use. Next, representative kits that find use in practicing the subject methods are further described.

Methods of Modulating Sebaceous Glands

As summarized above, the subject invention provides methods of modulating sebaceous glands in a host. In many embodiments, the methods include administering to a host an effective, amount of one or more active agents that modulate DGAT1 activity in the host to modulate, sebaceous gland activity in the host.

By DGAT1 activity is meant the activity of a DGAT1 protein, where representative DGAT1 proteins are disclosed in Cases et al., Proc. Nat'l Acad. Sci. USA (1998) 95:13018-13023 and Genbank Accession Nos.: AAC63997, AF059202; as well as U.S. Pat. Nos. 6,100,077 and 6,344,548 and the priority applications to the present application (listed above); the disclosures of which are herein incorporated by reference.

As DGAT1 activity is modulated in certain embodiments of the invention, DGAT1 activity is increased or decreased in these embodiments. In many embodiments, DGAT1 activity is increased or decreased by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, as compared to a baseline DGAT1 activity level, e.g., that observed in the host prior to administration of the active agent.

In some embodiments where the desired sebaceous gland modulation is a reduction in a sebaceous gland parameter, e.g., a reduction in sebum production, a reduction in sebaceous gland size, etc., one or more agents that decreases DGAT1 activity is administered to the host. For example, in certain embodiments, one or more agents that decreases DGAT1 activity is administered to the host. In these embodiments, the agent is typically a DGAT1 inhibitor.

In some embodiments where an increase in sebaceous gland activity is desired, one or more agents that increase DGAT1 activity is administered. For example, in certain embodiments, one or more agents that increases DGAT1 activity is administered to the host.

For the modulation of DGAT1 activity in a host, an effective amount of active agent(s) that modulates the activity, e.g. reduces the activity of DGAT1 in vivo, is administered to the host. The active agent may be a variety of different compounds, including: polynucleotide compositions (e.g., coding sequences, antisense compositions, siRNA compositions, etc.), polypeptide, including antibody, compositions, naturally occurring or synthetic small molecule compounds, etc.

In certain embodiments, the active agents administered to the host are polynucleotide to nucleic acid compositions. The nucleic acids may be coding sequences, e.g., genes, gene fragments etc., which may be present in expression vectors, where such vectors generally have convenient restriction sites located near the promoter sequence to provide for the insertion of nucleic acid sequences. Transcription cassettes may be prepared that include a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

In yet other embodiments of the invention, the active agent is an agent that modulates, and generally decreases or down regulates, the expression of DGAT1 in the host. Antisense molecules can be used to down-regulate expression of a gene in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-Q-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene,-expression. Ribozymes may be synthesized in vitro and administered to the patient or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

Alternatively, gene expression can be modified by gene silencing using double-strand RNA (Sharp (1999) Genes and Development 13: 139-141). RNAi, otherwise known as double-stranded RNA interference (dsRNAi) or small interfering RNA (siRNA), has been extensively documented in the nematode C. elegans (Fire, A., et al, Nature, 391, 806-811, 1998) and an identical phenomenon occurs in plants, in which it is usually referred to as post-transcriptional gene silencing (PTGS) (Van Blokland, R., et al., Plant J., 6: 861-877, 1994; deCarvalho-Niebel, F., et al., Plant Cell, 7: 347-358, 1995; Jacobs, J. J. M. R. et al., Plant J., 12: 885-893, 1997; reviewed in Vaucheret, H., et al., Plant J., 16: 651-659, 1998). The phenomenon also occurs in fungi (Romano, N. and Masino, G., Mol. Microbiol., 6: 3343-3353, 1992, Cogoni, C., et al., EMBO J., 15: 3153-3163; Cogoni, C. and Masino, G., Nature, 399: 166-169, 1999), in which it is often referred to as “quelling”. RNAi silencing can be induced many ways in plants, where a nucleic acid encoding an RNA that forms a “hairpin” structure is employed in most embodiments. Alternative strategies include expressing RNA from each end of the encoding nucleic acid, making two RNA molecules that will hybridize. Current strategies for RNAi induced silencing in plants are reviewed by Carthew et al (Curr Opin Cell Biol. 2001 13:244-8). RNAi is also described in WO 02/44321 and WO 01/68836; the priority documents of which are herein incorporated by reference.

Also of interest are polypeptide, e.g., proteinaceous, active agents. Specific polypeptide agents include proteins or active fragments thereof e.g., DGAT1 proteins, etc. A specific type of polypeptide active agent of interest is an antibody agent that modulates DGAT1 activity in the host. The antibodies may be monoclonal or polyclonal, and produced according to methods known in the art. Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.Consensus sequences of H and L J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. (1983) Mol. Cell. Bio. 3:280), Rous sarcoma virus LTR (Gorman et al. (1982) P.N.A.S. 79:6777), and moloney murine leukemia virus LTR (Grosschedl et aL. (1985) Cell 41:885); native Ig promoters, etc.

Naturally occurring or synthetic small molecule compounds of interest as active agents include numerous chemical classes, though typically they are organic molecules, preferably small organic compounds having a molecular weight of more than 50 and less than about 2,500 daltons. Candidate agents include functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Of particular interest are those agents identified by the screening assays of the subject invention, as described above.

In certain embodiments, in addition to (or in some embodiments instead of) the DGAT1 modulatory active agent, a leptin modulatory active agent, e.g., an agent that enhances or inhibits leptin activity, is administered. For example, in certain embodiments where a DGAT1 inhibitory agent is administered, a leptin activity enhancing agent may also be administered, such, that both a DGAT1 inhibitory agent and a leptin activity enhancing agent are administered to the host. Such embodiments include those embodiments where one wishes to modulate sebaceous glands in way that decreases a parameter thereof, e.g., reduces sebum production and/or reduces sebaceous gland size. In certain other embodiments, a leptin activity decreasing agent, e.g., a leptin activity inhibitor is administered to the host, either alone or in combination with a DGAT1 activity enhancing agent. Such embodiments include those embodiments where it is desired to increase a parameter of sebaceous glands, e.g., to increase sebum production and/or increase sebaceous gland size.

In practicing the subject methods, an effective amount of the active agent is administered to the host, where the term “effective amount” means a dosage sufficient to produce a desired result, where the desired result is the desired modulation, e.g., enhancement, reduction, of DGAT1 activity.

In practicing the subject methods, the active agent or agents are typically administered to the host in a physiologically acceptable delivery vehicle, e.g., as a pharmaceutical preparation. A variety of representative formulations, dosages, routes of administration for candidate agents, nucleic acid delivery vehicles and nucleic acid formulations for nucleic acid delivery are described below.

Formulations, Dosages, and Routes of Administration

The invention provides formulations, including pharmaceutical formulations, that include an agent which modulates sebaceous glands in a host. In general, a formulation comprises an effective amount of an agent that modulates DGAT1 (and/or leptin) activity in a host. An “effective amount” refers to an amount that is sufficient to produce a desired result, e.g., reduction or increase in a level of DGAT1 expression and/or activity, decrease in hair loss or hair sheen, an increase in thermotolerance, or skin drying etc. In many embodiments, the desired result is at least a reduction or increase in a phenotype as compared to a control such that the phenotype is more similar to normal.

Formulations

In the subject methods, the active agent(s) may be administered to the host using any convenient means capable of resulting in the desired reduction in of a sebaceous gland-related phenotype.

Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents; preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Other modes of administration will also find use with the subject invention. For instance, an agent of the invention can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), preferably about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

An agent of the invention can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985; Remington: The Science and Practice of Pharmacy, A. R. Gennaro, (2000) Lippincott, Williams & Wilkins. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Dosages

Although the dosage used will vary depending on the clinical goals to be achieved, a suitable dosage range is one which provides up to about 1 μg to about 1,000 μg or about 10,000 μg of an agent that reduces a symptom of a sebaceous gland disorder, or a sebaceous gland activity in a subject animal.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

Routes of Administration

Conventional and pharmaceutically acceptable routes of administration include intranasal, intramuscular, intratracheal, intratumoral, subcutaneous, intradermal, topical application, intravenous, rectal, nasal, oral and other parenteral routes of administration. Routes of administration may be combined, if desired, or adjusted depending upon the agent and/or the desired effect. The composition can be administered in a single dose or in multiple doses.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as an sebaceous gland disorder and psychological trauma associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A subject polynucleotide can be delivered as a naked polynucleotide, or associated with (complexed with) a delivery vehicle. “Associated with”, or “complexed with”, encompasses both covalent and non-covalent interaction of a polynucleotide with a given delivery vehicle.

Nucleic Acid Delivery Vehicles

In certain embodiment, an agent is a nucleic acid. Nucleic acids may be delivered using several different vehicles, including viral and non-viral delivery vehicles.

Viral Delivery Vehicles

A subject polynucleotide can be associated with viral delivery vehicles. As used herein, a “viral delivery vehicle” intends that the polynucleotide to be delivered is encapsidated in a viral particle.

Numerous viral genomes useful in in vivo transformation and gene therapy are known in the art, or can be readily constructed given the skill and knowledge in the art. Included are replication competent, replication deficient, and replication conditional viruses. Viral vectors include adenovirus, mumps virus, a retrovirus, adeno-associated virus, herpes simplex virus (HSV), cytomegalovirus (CMV), vaccinia virus, and poliovirus, and non-replicative mutants/variants of the foregoing. In some embodiments, a replication-deficient virus is capable of infecting slowly replicating and/or terminally differentiated cells, since the respiratory tract is primarily composed of these cell types. For example, adenovirus efficiently infects slowly replicating and/or terminally differentiated cells. In some embodiments, the viral genome itself, or a protein on the viral surface, is specific or substantially specific for cells of the targeted cell. A viral genome can be designed to be target cell-specific by inclusion of cell type-specific promoters and/or enhancers operably linked to a gene(s) essential for viral replication.

Where a replication-deficient virus is used as the viral genome, the production of virus particles containing either DNA or RNA corresponding to the polynucleotide of interest can be produced by introducing the viral construct into a recombinant cell line which provides the missing components essential for viral replication and/or production. Preferably, transformation of the recombinant cell line with the recombinant viral genome will not result in production of replication-competent viruses, e.g., by homologous recombination of the viral sequences of the recombinant cell line into the introduced viral genome. Methods for production of replication-deficient viral particles containing a nucleic acid of interest are well known in the art and are described in, for example, Rosenfeld et al., Science 252:431-434, 1991 and Rosenfeld et al., Cell 68:143-155, 1992 (adenovirus); U.S. Pat. No. 5,139,941 (adeno-associated virus); U.S. Pat. No. 4,861,719 (retrovirus); and U.S. Pat. No. 5,356,806 (vaccinia virus). Methods and materials for manipulation of the mumps virus genome, characterization of mumps virus genes responsible for viral fusion and viral replication, and the structure and sequence of the mumps viral genome are described in Tanabayashi et al., J. Virol. 67:2928-2931, 1993; Takeuchi et al., Archiv. Virol., 128:177-183, 1993; Tanabayashi et al., Virol. 187:801-804, 1992;,Kawano et al., Virol., 179:857-861, 1990; Elango et al., J. Gen. Virol. 69:2893-28900, 1988.

Non-Viral Delivery Vehicles

A subject polynucleotide can be administered using a non-viral delivery vehicle. “Non-viral delivery vehicle” (also referred to herein as “non-viral vector”) as used herein is meant to include chemical formulations containing naked or condensed polynucleotides (e.g, a formulation of polynucleotides and cationic compounds (e.g., dextran sulfate)), and naked or condensed polynucleotides mixed with an adjuvant such as a viral particle (i.e., the polynucleotide of interest is not contained within the viral particle, but the transforming formulation is composed of both naked polynucleotides and viral particles (e.g., adenovirus particles) (see, e.g., Curiel et al. 1992 Am. J. Respir. Cell Mol. Biol. 6:247-52)). Thus “non-viral delivery vehicle” can include vectors composed of polynucleotides plus viral particles where the viral particles do not contain the polynucleotide of interest. “Non-viral delivery vehicles” include bacterial plasmids, viral genomes or portions thereof, wherein the polynucleotide to be delivered is not encapsidated or contained within a viral particle, and constructs comprising portions of viral genomes and portions of bacterial plasmids and/or bacteriophages. The term also encompasses natural and synthetic polymers and co-polymers. The term further encompasses lipid-based vehicles. Lipid-based vehicles include cationic liposomes such as disclosed by Felgner et al (U.S. Pat. Nos. 5,264,618 and 5,459,127; PNAS 84:7413-7417, 1987; Annals N.Y. Acad. Sci. 772:126-139, 1995); they may also consist of neutral or negatively charged phospholipids or mixtures thereof including artificial viral envelopes as disclosed by Schreier et al. (U.S. Pat. Nos. 5,252,348 and 5,766,625).

Non-viral delivery vehicles include polymer-based carriers. Polymer-based carriers may include natural and synthetic polymers and co-polymers. Preferably, the polymers are biodegradable, or can be readily eliminated from the subject. Naturally occurring polymers include polypeptides and polysaccharides. Synthetic polymers include, but are not limited to, polylysines, and polyethyleneimines (PEI; Boussif et al., PNAS 92:7297-7301, 1995) which molecules can also serve as condensing agents. These carriers may be dissolved, dispersed or suspended in a dispersion liquid such as water, ethanol, saline solutions and mixtures thereof. A wide variety of synthetic polymers are known in the art and can be used. “Non-viral delivery vehicles” further include bacteria. The use of various bacteria as delivery vehicles for polynucleotides has been described. Any known bacterium can be used as a delivery vehicle, including, but not limited to non-pathogenic strains of Staphylococcus, Salmonella, and the like.

Formulations for Nucleic Acid Delivery

The polynucleotide to be delivered can be formulated as a DNA- or RNA-liposome complex formulation. Such complexes comprise a mixture of lipids which bind to genetic material (DNA or RNA) by means of cationic charge (electrostatic interaction). Cationic liposomes which may be used in the present invention include 3β-[N-(N′, N′-dimethyl-aminoethane)-carbamoyl]-cholesterol (DC-Chol), 1,2-bis(oleoyloxy-3 -trimethylammonio-propane (DOTAP) (see, for example, WO 98/07408), lysinylphosphatidylethanolamine (L-PE), lipopolyamines such as lipospermine, N-(2-hydroxyethyl)-N,N-dimethyl-2,3-bis(dodecyloxy)-1-propanaminium bromide, dimethyl dioctadecyl ammonium bromide (DDAB), dioleoylphosphatidyl ethanolamine (DOPE), dioleoylphosphatidyl choline (DOPC), N(1,2,3-dioleyloxy) propyl-N,N,N-triethylammonium (DOTMA), DOSPA, DMRIE, GL-67, GL-89, Lipofectin, and Lipofectamine (Thiery et al. (1997) Gene Ther. 4:226-237; Felgner et al., Annals N.Y. Acad. Sci. 772:126-139, 1995; Eastman et al., Hum. Gene Ther. 8:765-773, 1997). Polynucleotide/lipid formulations described in U.S. Pat. No. 5,858,784 can also be used in the methods described herein. Many of these lipids are commercially available from, for example, Boehringer-Mannheim, and Avanti Polar Lipids (Birmingham, Ala.). Also encompassed are the cationic phospholipids found in U.S. Pat. Nos. 5,264,618, 5,223,263 and 5,459,127. Other suitable phospholipids which may be used include phosphatidylcholine, phosphatidylserine, phosphatidylethanolamine, sphingomyelin, phosphatidylinositol, and the like. Cholesterol may also be included.

Utility

The subject compositions and methods of modulating the activity of a sebaceous gland find use in a variety of therapeutic protocols. In general, these protocols involve administering to a host suffering from a sebaceous gland condition an effective amount of one or more active agents that modulate DGAT1 (and/or leptin) function in the host to modulate sebaceous gland parameters (e.g., sebum production, size, etc.) in the host and treat the host for the condition.

By treatment is meant at least an amelioration of a symptom associated with the pathological condition afflicting the host, where amelioration is used in a broad sense to refer to at least a reduction in the magnitude of a parameter, e.g. symptom, associated with the pathological condition being treated, such as hair greasiness, number and size of comidones, etc. As such, treatment also includes outcomes where the pathological condition, or at least symptoms associated therewith, are completely inhibited, e.g. prevented from happening, or stopped, e.g. terminated, such that the host no longer suffers from the pathological condition, or at least the symptoms that characterize the pathological condition. For example, where the disease condition is marked by the presence of elevated hair greasiness, treatment includes at least a reduction in the observed hair greasiness, including a restoration of normal hair greasiness.

A variety of hosts are treatable according to the subject methods. Generally such hosts are mammals or mammalian, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Of particular interest is treatment and prevention of sebaceous gland disorders associated with undesirable sebaceous gland activity, including overactive sebaceous glands, underactive sebaceous glands, mal-developed sebaceous glands, blocked sebaceous glands, infected sebaceous glands, inflamed sebaceous glands and the like. Examples of sebaceous gland disorders include, but are not limited to: acne, including open comedos (blackheads) and whiteheads, pimples, deep acne, acne conglobata, comedos, cysts, microcomedos, papules, Propionibacterium acnes (P. acnes) infections, pustules and acne vulgaris, acne rosacea, acne conglobata, perioral dermatitis, sebaceous cysts, primary seborrhea (seborrhea oleosa), secondary seborrhea (seborrhea sicca) and alopecia. Also of interest are disorders treatable by altering the function of a sebaceous gland, such as dandruff and dry skin, and “cosmetic” sebaceous gland disorders, including dry hair, greasy hair, hair and skin sheen and other minor cosmetic disorders of the skin and/or complexion. The subject methods also find use in the modulation of sebaceous gland activity in hosts not suffering from a particular sebaceous gland condition but in which the modulation of sebaceous gland activity is nonetheless desired.

In some embodiments, where a reduction of sebaceous gland activity is desired, one or more agents that decreases DGAT1 activity (and/or increases leptin activity) may be administered, whereas when an increase in sebaceous gland activity is desired, one or more agents that increases DGAT1 activity (and/or decreases leptin activity) may be administered.

Subject treatment methods are typically performed on hosts with such disorders or on hosts with a desire to avoid contracting such disorders. Subjects of particular interest include those that are prone to sebaceous gland disorders, such as human teenagers or adolescents, typically aged between 12 and 17 years.

The invention also includes preventing or reducing the risk of a sebaceous gland disorder in a host by administering a pharmaceutical composition.

Kits

Also provided by the subject invention are kits for practicing the subject methods, as described above. The subject kits at least include one or more of a pharmaceutical preparation comprising at least one active agent that modulates DGAT1 activity (and/or leptin activity), as described above. Other optional components of the kit include: a syringe or another administration device. The various components of the kit may be present in separate containers or certain compatible components may be precombined into a single container, as desired. In many embodiments, kits with unit doses of the active agent, e.g. in oral or injectable doses, are provided. In many embodiments the subject composition is contained within a media, such as a hair shampoo, a hair conditioner, a soap bar, a facial scrub, a facial cream for topical administration and the like.

In addition to above-mentioned components, the subject kits typically further. include instructions for using the components of the kit to practice the subject methods treating a host suffering from a sebaceous gland condition by administering to said host an effective amount of one or more active agents that modulate DGAT1 (and/or leptin) activity in the host to modulate sebaceous gland activity in the host and treat the host for the condition. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Animal Models for Sebaceous Gland Disorders

The invention provides a non-human animal model for a sebaceous gland disorder. In general, the non-human animal model is characterized by having abnormal DGAT activity.

A non-human animal may be any animal, e.g., a mammal or avian species that can serve as an animal model for testing therapies for sebaceous gland conditions. In many embodiments the non-human animal is a laboratory animal, usually a rodent, e.g., mouse, rat, hamster, guinea pig or the like. Other suitable laboratory animals are rabbits, cats, dogs, small monkeys, and apes. In addition, certain farm animals are also often employed as laboratory animals, notably chickens, goats, sheep, and pigs. Mice suitable for use in the present invention can be produced from any of a variety of background strains including, but not necessarily limited to, the strains C.B-17, C3H, BALB/c, C57131/6, AKR, BA, B10, 129, etc. Non-human animals are readily available from researchers or commercial suppliers, such as Jackson Laboratories (Bar Harbor, Me.), Charles River Breeding Laboratories (Wilmington, Mass.), Taconic Farms (Germantown, N.Y.), to mention a few such suppliers.

DGAT activity, including DGAT1 and/or DGAT2 activity, may be modified in animals by a variety of methods. In many embodiments, these methods involve modifying the expression of DGAT, leptin or the leptin receptor in a transgenic animal. In many embodiments, the expression of a DGAT, leptin or the leptin receptor endogenous to the animal is reduced in an animal. This may be accomplished through knockout strategies, where an nucleic acid insertion into an endogenous gene inactivates the gene (described in U.S. Pat. Nos. 5,487,992; 5,627,059; 5,631,153; and 6,204,061), or by other methods e.g. antisense, inhibitory RNA (RNAi), ribozyme or co-supression technologies, as is known in the art (e.g. Hannon et al., Nature 418:244-51, 2002; Ueda, J Neurogenet. 15:193-204, 2001; Review. Lindenbach et al., Mol Cell. 9:925-7, 2002; Brantl, Biochim Biophys Acta. 1575:15-25, 2002; Zhang et al., Ann NY Acad Sci. 923:210-33, 2000). In other embodiments, an endogenous DGAT, leptin or the leptin receptor or an exogenous DGAT, leptin or the leptin receptor is over-expressed in an animal. In these embodiments, a DGAT, leptin or leptin receptor coding sequence (for example, a coding sequence provided by one of the following NCBI accession: NM_{—}010046 (SEQ ID NO: 1), XM_{—}035370 (SEQ ID NO:2), NM_{—}053437 (SEQ ID NO: 3), AJ318490 (SEQ ID NO: 4), AF221132 (SEQ ID NO:5), AF468649 (SEQ ID NO: 6), AY093657 (SEQ ID NO: 7), AF384161 (SEQ ID NO:8), NM_{—}012079 (SEQ ID NO: 9), AF384163 (SEQ ID NO: 10), AF384162 (SEQ ID NO:11), AF078752 (SEQ ID NO: 12), NM_{—}013076 (SEQ ID NO: 13), NM_{—}010704 (SEQ ID NO:14), NM_{—}008493 (SEQ ID NO: 15), BTU83512 (SEQ ID NO: 16), NM_{—}000230 (SEQ ID NO: 17), and NM_{—}012596 (SEQ ID NO: 18)) is cloned into an expression cassette in an appropriate vector, and transferred into the genome of an animal to make a transgenic animal. In many embodiments, the animal is homozygous for a defect in a gene selected from DGAT, obese (encoding leptin) or the leptin receptor. In many embodiments, the subject animal is homozygous for a knockout in one of these genes.

Cloning technology, cloning strategies, expression cassettes, and suitable vectors for performing animal transformation are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Methods of generating transgenic, non-human animals, particularly transgenic, non-human mammals, are also known in the art. See, e.g., U.S. Pat. Nos. 6,268,545; 6,255;554; 6,222,094; 5,387,742, 4,736,866 and 5,565,186 and 6,204,43; “Transgenic Animal Technology” C. A. Pinkert, ed. (1997) Acad. Press; “Gene Knockout Protocols” M. J. Tymms, et al., eds. (2001) Humana Press; and “Gene Targeting: A Practical Approach” A. L. Joyner, ed. (2000) Oxford Univ. Press.

One method for producing a transgenic animal employs embryonic stem (ES) cells obtained from an animal to be transformed, e.g. mouse, rat, guinea pig, etc. In these methods, ES cells are grown on an appropriate fibroblast-feeder layer or grown in the presence of appropriate growth factors, such as leukemia inhibiting factor (LIF). When ES cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for mutant cells having the construct. By providing for a different phenotype of the blastocyst and the ES cells, chimeric progeny can be readily detected. Progeny of transgenic animals may be screened for the presence of the modified gene and males and females having appropriate modified genomes are mated to produce homozygous progeny.

In certain embodiments, transgenic animals may be inter-crossed or may contain more than one genetic modification in order to produce a subject animal model. For example, an animal overexpressing DGAT1 may be bred with an animal knockout of a leptin-encoding gene to produce an a subject animal model containing an increase in DGAT activity and a decrease in leptin activity and an animal overexpressing leptin may also be bred with an animal knockout of a DGAT-encoding gene to produce an a subject animal model containing a decrease in DGAT activity and an increase in leptin activity, etc. Subject animals may also be intercrossed with hairless “nude” strains of animals, e.g. nude mice.

In embodiments where DGAT, leptin or the leptin receptor is overexpressed in a subject animal, DGAT, leptin or the leptin receptor expression is increased more than about 1.5-fold, more than about 2-fold, more than about 3-fold, more than about 5-fold, more than about 10-fold or even more than about 100-fold in a subject animal, as compared to an animal in which DGAT, leptin or the leptin receptor expression is not increased.

In embodiments where DGAT, leptin or the leptin receptor expression is decreased in a subject animal, DGAT, leptin or the leptin receptor expression is decreased by more than about 30%, more than about 50%, more than about 70%, more than about 90%, more than about 95% or even more than about 98%, about 99% or 99.5% in a subject animal, as compared to an animal in which DGAT, leptin or the leptin receptor expression is not decreased.

The subject animals have abnormal sebaceous gland activity. In certain embodiments, the phenotypes exhibited by the subject animals include, but are not limited to, dry fur, fur with reduced sheen, fur loss, altered sebaceous gland development, atrophic sebaceous glands, altered sebaceous gland activity, altered fur lipid abnormalities and impaired water repulsion and defective thermoregulation after water immersion as compared to normal animals. In most embodiments the subject animal is characterized as having abnormal DGAT activity, such as an increase or decrease in DGAT activity relative to a normal animal of the same species. Such animals find use in a variety of applications, including the screening methods described below.

### Screening Assays

The invention provides methods of screening a candidate agent for sebaceous gland modulatory activity, e.g. stimulators or inhibitors of sebaceous gland activity. These screening assays typically provide for qualitative/quantitative measurements of a phenomenon associated with sebaceous glands in the presence of a particular candidate agent. The screening methods be performed in vivo, ex vivo, in vitro or in a cell free system, which are readily developed by those of skill in the art. Test agents that have a desirable effect in any subject screening assay method find use in a variety of applications, including modulating sebaceous gland activity in a host or treating a sebaceous gland disorder.

Using the above screening methods, a variety of different agents may be identified. Such agents may target the DGAT enzyme itself, or an expression regulatory factor thereof Such agents may be inhibitors or promoters of DGAT activity, where inhibitors are those agents that result in at least a reduction of DGAT activity as compared to a control and enhancers result in at least an increase in DGAT activity as compared to a control. Such agents may alternatively target leptin itself, or an expression regulatory factor thereof. Such agents may be inhibitors or promoters of leptin activity, where inhibitors are those agents that result in at least a reduction of leptin activity as compared to a control and enhancers result in at least an increase in leptin activity as compared to a control.

Specific screening assay methods are described below.

In Vivo Assays

The invention provides in vivo methods of screening a candidate agent for sebaceous gland modulatory activity. In general, the method involves administering a candidate agent to a subject transgenic animal and determining the effect of the candidate agent on the activity of sebaceous glands of the transgenic animal in order to assess the candidate agent's sebaceous gland modulatory activity.

In many embodiments the determining step of the in vivo assay method involves measuring a phenomenon associated with sebaceous glands, including DGAT activity, DGAT expression, lipid (e.g. TAG) biosynthesis, deposition or secretion and the like, performing visual examination of sebaceous glands for visual indications, such a change in sebaceous gland size, shape, color, inflammation, the formation of comidones, or determining the effect of water repulsion, hair sheen, thermoregulation or body weight after water immersion, or hair drying, hair dryness and the like.

In vivo assays of the invention include controls, where suitable controls include a sample in the absence of the test agent. Generally a plurality of assay mixtures is run in parallel with different agent concentrations to obtain a differential response to the various concentrations. Typically, one of these concentrations serves as a negative control, i.e. at zero concentration or below the level of detection.

A candidate agent of interest is one that modulates, i.e., reduces or increases, DGAT activity, DGAT expression, lipid (e.g. TAG) biosynthesis, deposition or secretion, hair sheen, hair lipids sebaceous gland size, water repulsion, or thermoregulation, etc., by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the test agent. In general, a candidate agent will cause a subject animal to be more similar to an equivalent animal that is not altered in DGAT activity.

Ex Vivo Assays

The invention provides ex vivo methods of screening a candidate agent for sebaceous gland modulatory activity. In general, the methods involve contacting a candidate agent with an isolated tissue having abnormal DGAT activity and determining the, effect of the candidate agent on a phenomenon associated with sebaceous glands in order to assess the candidate agent's sebaceous gland modulatory activity.

In many embodiments, a tissue with abnormal DGAT activity, particularly a skin tissue, is isolated from an animal. Methods of culturing isolated skin tissue ex vivo are known in the art (e.g. Companjen et al., Arch Dermatol Res. 2001 293:184-90; Calabrese et al., Drugs Exp Clin Res. 1999 25:43-9). In many embodiments the subject tissue is a tissue from a subject model animal.

In may embodiments, the test agent is applied to culture media, or directly applied, usually topically, to the ex vivo cultured tissue.

In many embodiments the determining step of the in vitro assay method involves measuring a phenomenon associated with sebaceous glands, including measureing DGAT activity, DGAT expression, lipid (e.g. TAG) biosynthesis, deposition or secretion and the like, or performing visual examination of sebaceous glands for visual indications, such a change in sebaceous gland size, shape, color, inflammation, or the formation of comidones. Hair characteristics, such as hair lipid compositions, sheen and dryness may also be determined.

Ex vivo assays of the invention include controls, where suitable controls include a sample in the absence of the test agent. Generally a plurality of assay mixtures is run in parallel with different agent concentrations to obtain a differential response to the various concentrations. Typically, one of these concentrations serves as a negative control, i.e. at zero concentration or below the level of detection.

A candidate agent of interest is one that modulates, i.e., reduces or increases, DGAT activity, DGAT expression, lipid (e.g. TAG) biosynthesis, deposition or secretion, hair sheen, hair lipids sebaceous gland size etc., by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the test agent. In general, a candidate agent will cause a subject tissue to be more similar to an equivalent tissue that is not altered in DGAT activity.

In Vitro Assays

The invention provides in vitro methods of screening a candidate agent for sebaceous gland modulatory activity. In general, the methods involve contacting a cell with abnormal DGAT activity with a candidate agent and determining the effect of the agent on the cell in order to assess the candidate agent's sebaceous gland modulatory activity.

In many embodiments, the cell with abnormal DGAT activity is an in which DGAT gene expression has been modified as compared to an unaltered cell. Methods for altering gene expression in a cell are well known to one of skill in the art (discussed in Ausubel, et al, Short Protocols in Molecular Biology, 3rd ed., Wiley & Sons, 1995; Sambrook, et al., Molecular Cloning. A Laboratory Manual, Second Edition, (1989) Cold Spring Harbor, N.Y.). These methods may involve DGAT overexpression via introduction of a genetic construct designed to express DGAT coding sequences, or may involve downregulating DGAT expression via knockout strategies (described in U.S. Pat. Nos. 5,487,992; 5,627,059; 5,631,153; and 6,204,061), or by other methods e.g. antisense, inhibitory RNA (RNAi), ribozyme or co-supression technologies, as is known in the art (e.g. Hannon et al., Nature 418:244-51, 2002; Ueda, J Neurogenet. 15:193-204, 2001; Review. Lindenbach et al., Mol Cell. 9:925-7, 2002; Brantl, Biochim Biophys Acta. 1575:15-25, 2002; Zhang et al., Ann NY Acad Sci. 923:210-33, 2000).

In embodiments where DGAT is overexpressed in a cell, DGAT expression is increased more than about 1.5-fold, more than about 2-fold, more than about 3-fold, more than about 5-fold, more than about 10-fold or even more than about 100-fold in the cell, as compared to an cell in which DGAT is not increased.

In embodiments where DGAT expression is decreased in a cell, DGAT expression is decreased by more than about 30%, more than about 50%, more than about 70%, more than about 90%, more than about 95% or even more than about 98%, about 99% or 99.5% in the, as compared to a cell in which DGAT is not decreased.

In many embodiments the subject cell is a cell from a subject model animal. In these embodiments, a cell, particularly a skin cell, from a subject animal model is isolated and may be cultured to produce a cell that has altered DGAT activity. In certain embodiments the subject skin cell is a sebocyte cell, isolation and culture methods for which are known in the art (Rosenfield et al., In Vitro Cell Dev Biol Anim 2002 38:54-7; Rosenfield et al., J Invest Dermatol. 1999 112:226-32).

In many embodiments the determining step of the in vitro assay method involves measuring a phenomenon associated with sebaceous glands, including DGAT activity, DGAT expression, lipid (e.g. TAG) biosynthesis, deposition or secretion and the like,

In vitro assays of the invention include controls, where suitable controls include a sample in the absence of the test agent. Generally a plurality of assay mixtures is run in parallel with different agent concentrations to obtain a differential response to the various concentrations. Typically, one of these concentrations serves as a negative control, i.e. at zero concentration or below the level of detection.

A test agent of interest is one that modulates, i.e., reduces or increases, DGAT activity, DGAT expression, lipid (e.g. TAG) biosynthesis, deposition or secretion and the like, by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the test agent. In general, a test agent will cause a subject cell to be more similar to an equivalent cell that is not altered in DGAT activity.

Cell Free Assays

The invention provides cell free methods of screening a candidate agent for sebaceous gland modulatory activity. In general, the methods involve admixing an extract of a cell (or a synthetic mimetic thereof) with abnormal DGAT activity with a candidate agent and determining the effect of the agent on the extract in order to assess the candidate agent's sebaceous gland modulatory activity. In many embodiments the assay methods involve measuring DGAT activity, lipid (e.g. TAG) biosynthesis, or and the like,

Cell free assays of the invention include controls, where suitable controls include a sample in the absence of the candidate agent. Generally a plurality of assay mixtures is run in parallel with different agent concentrations to obtain a differential response to the various concentrations. Typically, one of these concentrations serves as a negative control, i.e. at zero concentration or below the level of detection.

A test agent of interest is one that modulates, i.e., reduces or increases, DGAT activity, lipid biosynthesis or the like, by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the test agent. In general, a candidate agent will cause a subject extract to be more similar to an equivalent extract from a cell that is not altered in DGAT activity.

A variety of other reagents may be included ini the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used.

Candidate Agents

The terms “candidate agent,” “test agent,” “agent”, “substance” and “compound” are used interchangeably herein and describe a variety of agents that may be screened using the above methods.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. New potential therapeutic agents may also be created using methods such as rational drug design or computer modeling. For example, numerous means are available for random and directed synthesis of a wide variety of organic compounds and biomolecules, including expression of randomized oligonucleotides and oligopeptides.

Screening may be directed to known pharmacologically active compounds and chemical analogs thereof, or to new agents with unknown properties such as those created through rational drug design.

A variety of other reagents may be included in screening assays. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, anti-microbial agents, etc. may be used. The mixture of components is added in any order that, provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hour will be sufficient.

Candidate agents may also include biopolymers, including nucleic acids (e.g. DNA, RNA, cDNA, plasmids and this like), for example those encoding DGAT1 or DGAT2, leptin, leptin receptor, antisense DGAT1 or DGAT2, leptin, leptin receptor nucleic acids and the like), carbohydrates, lipids (e.g. lipids that inhibit the activity of DGAT) and proteins and polypeptides, (such as DGAT1 or DGAT2, leptin, leptin receptor or an antibody specific for DGAT1 or DGAT2, leptin, leptin receptor, etc.).

In particular embodiments, the candidate agent may be niacin, or other agents known in the art, e.g. those described in Lesnik et al. (Arch Dermatol Res 1992;284(2):100-5).

Agents that have an effect in an assay method of the invention may be further tested for cytotoxicity, bioavailability, and the like, using well known assays. Agents that have an effect in an assay method of the invention may be subjected to directed or random and/or directed chemical modifications, such as acylation, alkylation, esterification, amidification, etc. to produce structural analogs. Such structural analogs include those that increase bioavailability, and/or reduced cytotoxicity. Those skilled in the art can readily envision and generate a wide variety of structural analogs, and test them for desired properties such as increased bioavailability and/or reduced cytotoxicity and/or ability to cross the blood-brain barrier.

The following examples are presented for purposes of illustration only, and are not to be construed as limiting on the scope of the invention in any way.

### EXPERIMENTAL

Materials and Methods

Mice: Dgat^{-/-} mice in C57BL/6 background were generated and genotyped as described (Smith, et al.(2000) Nat. Genet. 25:87-90). Wild-type (Dgat^{+/+}), ob/+, and Agouti yellow (A^{Y}/a) mice (all in C57BL/6 background) were from The Jackson Laboratory (Bar Harbor, Me., USA). DGAT1 deficiency was introduced into ob/ob and A^{Y}/a mice through breeding. Ob/ob mice lack leptin and, as a result, are obese and diabetic. A^{Y}/a mice are obese because of the ectopic production of agouti-signaling protein, which antagonizes the effects of melanocyte-stimulating hormone in the hypothalamus. A^{Y}/a mice have a functional leptin pathway, although they are leptin-resistant. Mice were housed in a pathogen-free barrier facility (12-hour light/dark cycle) and fed rodent chow (Ralston Purina Co., St. Louis, Mo., USA).

Water repulsion and temperature measurements: Mice were immersed in 37° C. water for 3 minutes and placed on a paper towel for about 5 seconds to absorb excess water. The mice were then exposed to ambient temperature (about 20° C.), and their weights and temperatures were recorded for 30-60 minutes. Core body temperature was measured rectally with a digital thermometer (model 4600; Yellow Springs Instruments Co., Yellow Springs, Ohio, USA).

Leptin infusion and testosterone administration: For peripheral (subcutaneous) infusion, a micro-osmotic pump (DURECT Corp., Cupertino, Calif., USA) was implanted in the interscapular region. The pump delivered recombinant human leptin (a gift from F. Chehab, University of California, San Francisco) at 250 ng/h for 14 days. This dose restores a normal plasma leptin level in mice with leptin deficiency resulting from lipodystrophy. For central (intracerebroventricular) infusion, a cannula (Brain Infusion Kit II, DURECT Corp.) was attached to the implanted micro-osmotic pump, and the needle was inserted 0.5 mm caudal and 1 mm lateral to the bregma. Leptin (10 ng/h) was infused for 14 days. This infusion rate does not affect plasma leptin concentrations. Testosterone propionate (Sigma Chemical Co., St. Louis, Mo., USA) was dissolved in vegetable oil and injected subcutaneously.

In situ hybridization: In situ hybridization was performed as described (Meiner, V. et al.(1997). J. Lipid Res. 38:1928-1933). Briefly, skin sections from wild-type mice were deparaffinized and fixed in 4% paraformaldehyde. After proteinase K digestion, the sections were hybridized at 55° C. for 12 hours with ³⁵S-labeled antisense or sense DGAT1 RNA probes. The sections were washed for 20 minutes in 2×SSC, 10 mM β-mercaptoethanol, and 1 mM EDTA, treated with RNase A (20 μg/ml), and washed at high stringency (0.1×SSC, 10 mM β-mercaptoethanol, and 1 mM EDTA) for 2 hours at 60° C. The sections were dehydrated, dipped in photographic emulsion NTB₂ (Eastman Kodak Co. Scientific Imaging Systems, Rochester, N.Y., USA), and stored at 4° C. After 8 weeks of exposure, the sections were developed and counterstained with hematoxylin and eosin.

Histology: Skin samples were fixed overnight in buffered formalin and embedded in paraffin. Sections were stained with hematoxylin and eosin.

Extraction and analysis of fur lipids: Hair lipids were extracted as follows. Briefly, about 150 mg of fur was clipped from the back of the mouse and treated twice with 20 ml of acetone for 15 minutes. Lipid extracts were filtered, dried under N₂, and resuspended in chloroform. Samples (150 μg of the dried lipids) were loaded on a TLC plate (Silica Gel 60; Sigma-Aldrich, St. Louis, Mo., USA) and resolved with hexane/ethyl ether/acetic acid (80:20:1 vol/vol/vol) or hexane/benzene (55:45 vol/vol). The latter system allowed a better separation of nonpolar lipids. For transesterification, the lipid was scraped from the TLC plate, incubated with methanolic acid/toluene (4:1 vol/vol) at 37° C. for 12 hours, and extracted twice with hexane (1.5 ml). For visualization of lipids, the TLC plate was either exposed to iodine vapor or sprayed with cupric sulfate (3%)/phosphoric acid (8%) and charred at 150° C. for color development. For 1,2-diol staining, the plate was sprayed with 1% lead tetraacetate (dissolved in benzene), followed by 0.05% pararosaniline (dissolved in acetic acid/acetone, 1:9 vol/vol).

Real-time PCR: Skin was homogenized, and total RNA was extracted (RNA STAT; Tel-Test Inc., Friendswood, Tex., USA). Primer and probe sequences (actin forward 5′-CATCTTGGCCTCACTGTCCA-3′ (SEQ ID NO: 19), reverse primer: 5′-GGGCCGGACTCATCGTACT-3′ (SEQ ID NO: 20), probe: 5′-CTTCCAGCAGATGTGGATCAGCAAGC-3′ (SEQ ID NO: 21); DGAT2 forward primer: 5′-AGTGGCAATGCTATCATCATCGT-3′ (SEQ ID NO: 22), reverse primer: 5′-AAGGAATAAGTGGGAACCCAGATCA-3′ (SEQ ID NO: 23), probe: 5′-CCTGG-CAAGAACGCAGTCACCCTG-3′ (SEQ ID NO: 24)) were selected with Primer Express software (Perkin-Elmer Applied Biosystems, Foster City, Calif., USA). RNA (1 μg) was reverse-transcribed in a 20-μl reaction containing oligo (dT)₁₂₋₁₈ primer and Superscript II enzyme (Invitrogen Corp., Carlsbad, Calif., USA). Each PCR (50 μl) contained 1 μl of cDNA, 1×gold buffer II, 4 mM MgCl₂, 500 μM dNTP, primers (200 nM), 100 nM probe (labeled with 6-carboxyfluorescein), and 1.25 U AmpliTaq Gold DNA polymerase (Perkin-Elmer Applied Biosystems). Real-time PCR was performed and analyzed with the ABI Prism 7700 Sequence Detection System (Perkin-Elmer Applied Biosystems). Relative expression levels were calculated by the comparative C_{T} (cycle of threshold detection) method as outlined in the manufacturer's technical bulletin; β-actin expression was used as control.

Statistical analysis: Data are expressed as mean±SD. Differences in weight and temperature curves were compared by ANOVA followed by the Tukey-Kramer test, as appropriate.

### EXAMPLE I

### Preparation and Characterization of DGAT Knockout Mice

DGAT knockout mice were generated using standard techniques of gene targeting. A mouse P1 clone containing the mouse DGAT gene was isolated from a genomic 129/Sv library. Short and long arms of homologous sequences were amplified by PCR from this clone and subcloned intopNTKLoxP to generate a gene targeting vector. The vector contained a neomycin resistance gene for positive selection and a thymidine kinase gene for negative selection. Upon homologous recombination, the vector was designed to interrupt the DGAT coding sequences at amino acid 360 of the 498-amino acid murine protein. The entire C-terminus, including a highly conserved region common to all ACAT gene family members is deleted. The gene targeting vector was electroporated into RF8 embryonic stem cells by electroporation, and several targeted clones were identified by Southern blotting (targeting frequency of˜1 in 300).

One of these targeted clones was injected into C57BL/6 blastocysts and chimeras were generated; male chimeras subsequently passed the DGAT knockout mutation through the germline to their offspring. The resultant mice, which were heterozygous for the DGAT gene disruption, were intercrossed to generate mice that were homozygotes.

Inactivation of the DGAT gene in the homozygote knockouts was verified by examining DGAT mRNA which was found to be absent in the knockout mice. In activation of the DGAT gene was also verified by studying DGAT activity in tissues using an assay that measures the incorporation of [14C]oleoyl CoA into triglycerides. The results from the activity assays show that DGAT activity is virtually gone from every nearly every tissue tested.

### EXAMPLE II

### Fur Abnormalities and Impaired Water Repulsion in Dgat^{31 /-} Mice

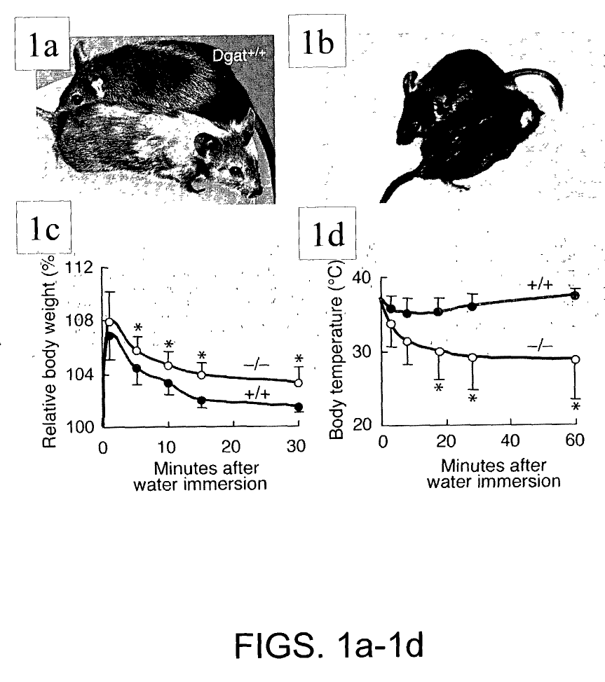

Dgat^{-/-} mice had normal fur appearance at weaning. After puberty (age 6-8 weeks), however, the fur of Dgat^{-/-} mice appeared drier and displayed a less prominent sheen than that of Dgat^{+/+} mice (FIG. 1a). Hair loss also occurred thereafter (FIG. 1a), beginning on the dorsal surface of the neck and proceeding caudally. Hair loss was more prominent in male mice than in female mice. Heterozygous (Dgat^{+/-}) mice appeared normal.

Because of their fur abnormalities, we tested the ability of Dgat^{-/-} mice to repel water and maintain normal body temperature when wet. Five minutes after water immersion, Dgat^{-/-} mice appeared wetter than Dgat^{+/+} mice, which were nearly dry (FIG. 1b). The delayed drying in Dgat^{-/-} mice resulted from increased water absorption during water immersion (FIG. 1c). Dgat^{-/-} mice became lethargic and exhibited little grooming behavior after water immersion, most likely because of hypothermia, which persisted for more than 60 minutes (FIG. 1d). Dry Dgat^{-/-} mice had no thermoregulatory defects. They had core body temperatures comparable to those of Dgat^{+/+} mice, both at room temperature (about 20° C.) and during prolonged cold exposure (24 hours at 4° C., not shown).

### EXAMPLE III

### Fur Abnormalities in DGAT1 -Deficient A^{Y}/a but not ob/ob Mice

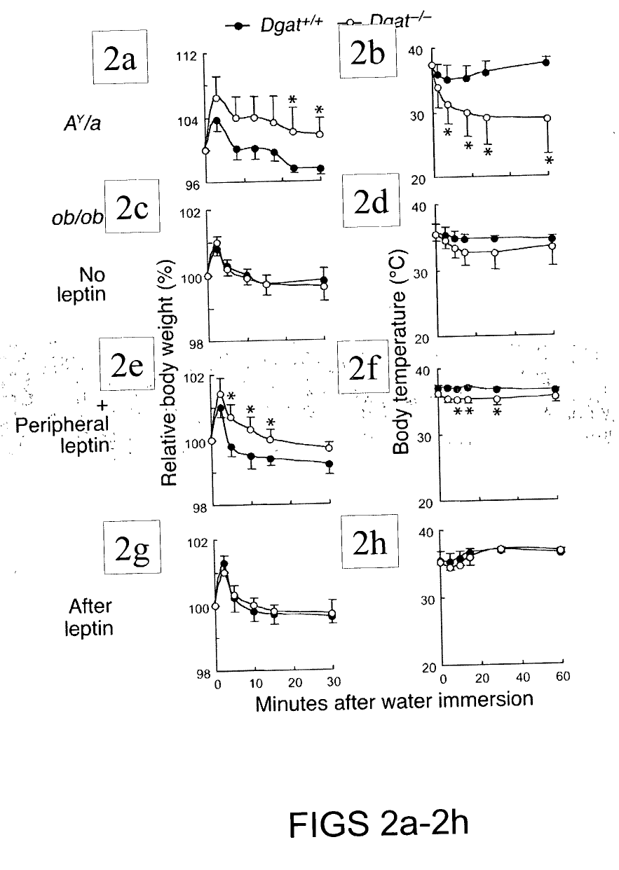

In a separate study to examine the metabolic effects of DGAT1 deficiency, we had introduced DGAT1 deficiency into two strains of genetically obese mice, ob/ob and A^{Y}/a. DGAT1 deficiency was associated with dry fur and hair loss in A^{Y}/a mice but had little impact on the fur of ob/ob mice. In addition, A^{Y}/a mice with DGAT1 deficiency (Dgat^{-/-} A^{Y}/a) retained more water than did wild-type (Dgat^{+/+}) A^{Y}/a mice (FIG. 2a) and developed hypothermia (FIG. 2b) after water immersion. In contrast, DGAT1 deficiency in ob/ob mice did not affect water repulsion (FIG. 2c) or thermoregulation (FIG. 2d).

To further explore whether the effects of DGAT1 deficiency on fur required leptin, we administered leptin to ob/ob mice with or without DGAT1 (Dgat^{+/+}ob/ob or Dgat^{-/-} ob/ob). After 2 weeks of continuous peripheral leptin infusion, Dgat^{-/-} ob/ob mice exhibited impaired water repulsion (FIG. 2e) and developed mild hypothermia (FIG. 2f) after water immersion. These findings were again absent 2 weeks after the cessation of leptin infusion (FIGS. 2, g and h).

### EXAMPLE IV

### Sebaceous Gland Atrophy in Dgat^{-/-} Mice

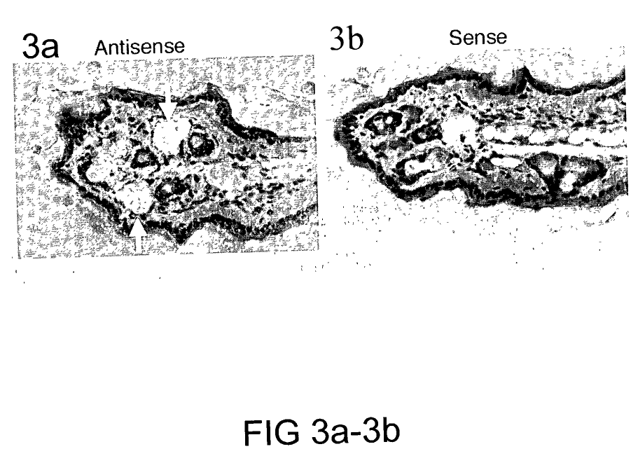

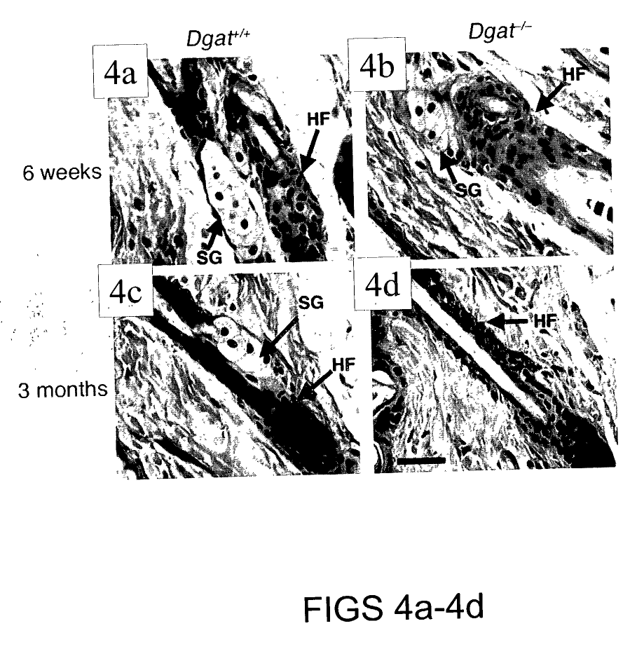

To investigate the function of DGAT1 in the skin, we examined DGAT1 expression by in situ hybridization, which revealed high DGAT1 mRNA levels in the sebaceous glands (FIG. 3). We therefore examined sebaceous gland morphology in Dgat^{-/-} mice. The sebaceous glands and hair follicles of young (6-week-old) Dgat^{-/-} mice appeared normal (FIGS. 4, a and b). In contrast, the skin of older (3-month-old) Dgat^{-/-} mice had atrophic sebaceous glands on both the ventral and the dorsal surfaces (FIGS. 4, c and d). For many hair follicles, no associated sebaceous glands could be identified.

### EXAMPLE V

### Atrophic Sebaceous Glands in DGAT1-Deficient A^{Y}/a Mice and Leptin-Treated ob/ob Mice

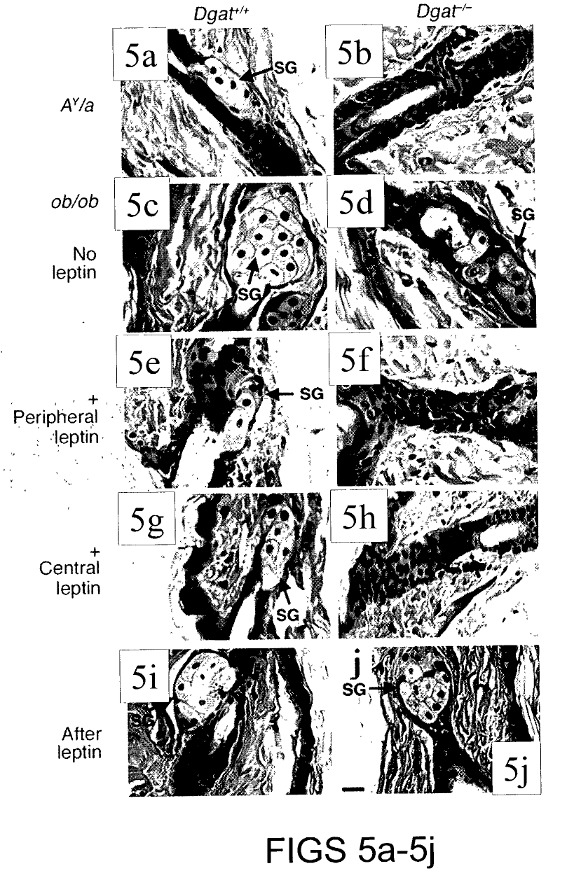

Similar to the situation for fur abnormalities, the sebaceous gland atrophy associated with DGAT1 deficiency was present in A^{Y}/a mice (FIGS. 5, a and b) but not in ob/ob mice (FIGS. 5, c and d). Dgat^{+/+} ob/ob mice, however, had larger sebaceous glands than did Dgat^{-/-} obob mice. Two weeks of peripheral or central leptin infusion decreased the size of sebaceous glands in Dgat^{+/+} ob/ob mice (FIGS. 5, e and g) but caused marked atrophy of sebaceous glands in Dgat^{-/-} ob/ob mice (FIGS. 5, f and h). These histological changes reverted to pretreatment states 2 weeks after the cessation of leptin administration (FIGS. 5, i and j).

### EXAMPLE VI

### Abnormal Fur Lipids in Dgat^{-/-} Mice

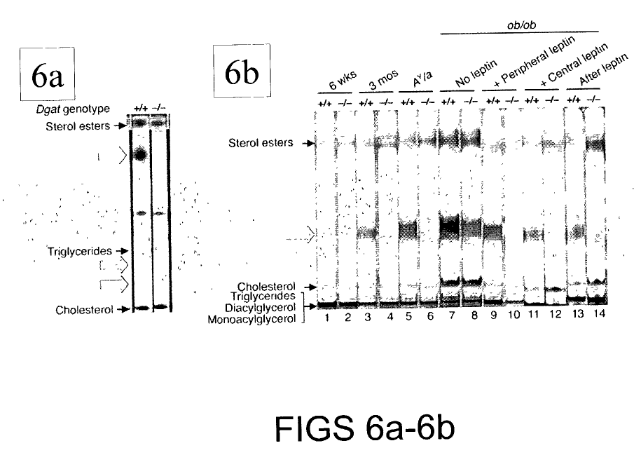

We analyzed the effects of DGAT1 deficiency on the composition of fur lipids, which are produced by sebaceous glands. In both Dgat^{+/+} and Dgat^{-/-} mice, the fur lipids contained sterol esters, free cholesterol, and triglycerides. In addition, the fur of adult Dgat^{+/+} mice contained several lipids that were lacking in the fur of adult Dgat^{-/-} mice (FIG. 6a). The most prominent of these missing lipids was slightly more polar than sterol esters. After transesterification, this lipid yielded two products—one migrated similarly to a fatty acid methyl ester standard, and the other contained a 1,2-diol group (not shown). Based on this result, as well as the migration and quantity of the lipid on TLC, it is most likely a type II wax diester, the most abundant component in murine fur lipids. This difference in fur lipid content was age-dependent: it was less striking in younger (6-week-old) mice (FIG. 6b, lanes 1 and 2) and more pronounced in adult (3-month-old) mice (FIG. 6b, lanes 3 and 4).

### EXAMPLE VII

### Abnormal Fur Lipids in DGAT1-Deficient A^{Y}/a Mice and Leptin-Treated ob/ob Mice

The fur of Dgat^{-/-} A^{Y}/a mice also contained little, if any, of the wax diester (FIG. 6b, lanes 5 and 6). In contrast, both Dgat^{+/+} ob/ob and Dgat^{-/-} ob/ob mice produced this fur lipid, although the quantity was slightly decreased in the fur of Dgat^{-/-} ob/ob mice (FIG. 6b, lanes 7 and 8). Two weeks of peripheral or central leptin administration had a minimal effect on the abundance of this fur lipid in Dgat^{+/+} ob/ob mice but caused a severe reduction in Dga^{-/-} ob/ob mice (FIG. 6b, lanes 9-12). Two weeks after the withdrawal of leptin, the fur of Dgat^{-/-} ob/ob mice contained this lipid again (FIG. 6b, lanes 13 and 14).

### EXAMPLE VIII

### Effects of Androgens on Fur Lipids in Dgat^{-/-} Mice

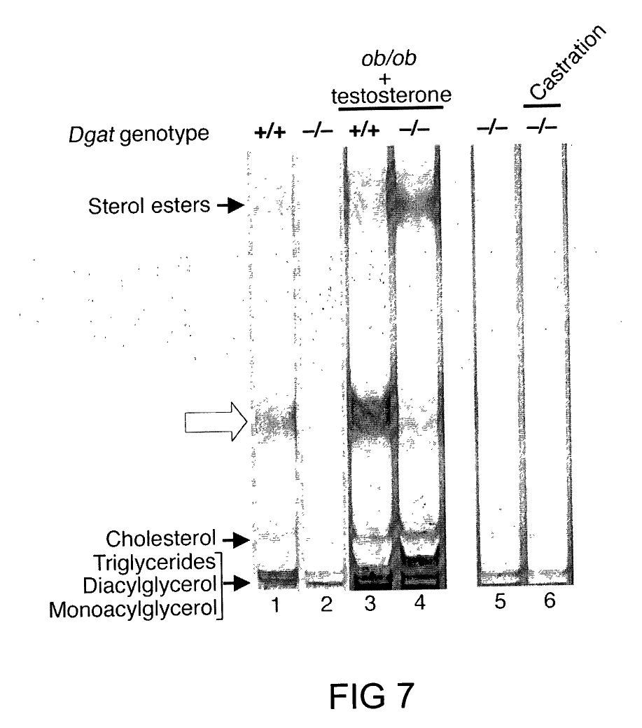

Because the effects of DGAT1 deficiency on the skin were most noticeable in postpubertal male mice, we investigated the role of androgens in mediating these effects. Because ob/ob mice have a defective hypothalamic-pituitary-gonadal axis, they do not undergo puberty and have decreased serum testosterone levels. This lack of normal testosterone production may ameliorate the effects of DGAT1 deficiency in ob/ob mice. To test this hypothesis, we injected both Dgat^{+/+} ob/ob and Dgat^{-/-} ob/ob male mice with a replacement dose of testosterone (5 μg/g body weight/day) and assessed its effects on fur lipid content. Two weeks of testosterone treatment did not eliminate the presence of wax diesters in the fur of Dgat^{-/-} ob/ob mice (FIG. 7, lanes 1-4). Testosterone replacement also did not cause atrophy of sebaceous glands in these mice (not shown).

To further explore whether androgens mediated the effects of DGAT1 deficiency in the skin, we castrated postpubertal Dgat^{-/-} male mice. Castration did not restore the normal production of fur lipids in Dgat^{-/-} male mice; rather, it completely eliminated the presence of wax diesters in their fur (FIG. 7, lanes 5 and 6). Castration also did not reverse the atrophy of sebaceous glands in these mice (not shown).

### EXAMPLE IX

### Upregulation of DGAT2 Expression in the Skin of DGAT1-Deficient ob/ob Mice

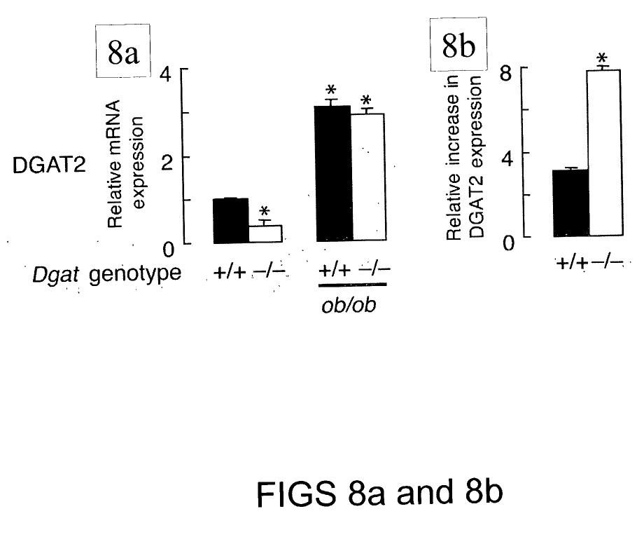

One possible mechanism by which the skin of Dgat^{-/-} ob/ob mice was largely protected from the effects of DGAT1 deficiency could be a compensatory increase in the expression of another DGAT enzyme. We therefore measured the mRNA expression of the recently identified DGAT2 to determine whether its expression was increased in the skin of Dgat^{-/-} ob/ob mice. In wild-type mice, DGAT2 mRNA was expressed highly in the skin (not shown). DGAT2 expression was not increased in Dgat^{-/-} mice and in fact was lower than in Dgat^{+/+} mice (FIG. 8a). However, leptin-deficient Dgat^{-/-} and Dgat^{+/+} mice had similarly increased levels of DGAT2 expression (FIG. 8a). Leptin deficiency, therefore, was associated with a greater upregulation of DGAT2 in the skin of Dgat^{-/-} mice than in that of Dgat^{+/+} mice (FIG. 8b).

It is evident from the above results and discussion that the subject invention provides an important new animal model for the treatment of sebaceous gland disorders, methods for treating sebaceous gland disorders and several sebaceous gland-related assay systems. As such, the subject methods and systems find use in a variety of different applications, including research, industry, and medicine. Accordingly, the present invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of modulating sebaceous glands in a host, said method comprising:
administering to said host an effective amount of a DGAT1 modulatory agent so that sebaceous glands in said host are modulated.

2. The method according to claim 1, wherein said sebaceous gland modulation comprises altered sebum production.

3. The method according to claim 1, wherein said sebaceous gland modulation comprises altered sebaceous gland size.

4. The method according to claim 1, wherein said DGAT1 modulatory agent is a DGAT1 inhibitory agent and said sebaceous gland modulation comprises at least one of reducing sebum production and reducing sebaceous gland size.

5. The method according to claim 1, wherein said host is a mammal.

6. The method according to claim 5, wherein said mammal is a human.

7. The method according to claim 1, wherein said DGAT1 modulatory agent is a DGAT1 activity enhancing agent and said sebaceous gland modulation comprises at least one of increasing sebum production and increasing sebaceous gland size.

8. The method according to claim 7, wherein said method further comprises administering a leptin activity enhancing agent to said host.

9. The method according to claim 1, wherein said agent is systemically administered to said host.

10. The method according to claim 1, wherein said agent is topically administered to said host.

11. A method of treating a host suffering from a sebaceous gland condition, said method comprising:
administering to said host an effective amount of a DGAT1 modulatory agent to treat said host for said condition.

12. The method according to claim 11, wherein said DGAT1 modulatory agent is a DGAT1 inhibitor.

13. The method according to claim 11, wherein said sebaceous gland condition is characterized by at least one of enhanced sebum production and sebaceous gland size as compared to a control.

14. The method according to claim 12, wherein said condition is chosen from acne, rosacea, perioral dermatitis, sebaceous cysts, seborrhea and alopecia.

15. The method according to claim 11, wherein said DGAT1 modulatory agent is a DGAT1 activity enhancing agent.

16. The method according to claim 15, wherein said method further comprises administering a leptin activity enhancing agent to said host.

17. The method according to claim 11, wherein said host is mammal.

18. The method according to claim 11, wherein said mammal is a human.

19. The method according to claim 11, wherein said agent is systemically administered to said host.

20. A pharmaceutical preparation comprising a DGAT1 activity modulatory agent in a pharmaceutically acceptable delivery vehicle.

21. The pharmaceutical preparation according to claim 20, wherein said DGAT1 activity modulatory agent is a DGAT1 activity inhibitor.

22. The pharmaceutical preparation according to claim 20, wherein said DGAT1 activity modulatory agent is a DGAT1 activity enhancer.

23. The pharmaceutical preparation according to claim 20, wherein said preparation further comprises a leptin activity modulatory agent.

24. The pharmaceutical preparation according to claim 20, wherein said preparation delivery vehicle is a systemic delivery vehicle.

25. The pharmaceutical preparation according to claim 20, wherein said delivery vehicle is a local delivery vehicle.

26. The pharmaceutical preparation according to claim 25, wherein said local delivery vehicle is a topical delivery vehicle.

27. A kit comprising:
(a) a pharmaceutical preparation comprising a DGAT1 activity modulatory agent in a pharmaceutically acceptable delivery vehicle; and
(b) instructions for treating a host suffering from a sebaceous gland condition by administering to said host an effective amount of said pharmaceutical preparation.

28. The kit according to claim 27, wherein said DGAT1 activity modulatory agent is a DGAT1 inhibitor.

29. The kit according to claim 28, wherein said pharmaceutical preparation further comprises a leptin activity modulatory agent.
